SEQUENCE LISTING (81 sequences)

SEQ ID NO: 1 - DNA, 59 bp, synthetic construct
  source 1..59: mol_type = other DNA
actctttccctacacgacgctcttccgatcgatcggaagagcacacgtctgaactccag

SEQ ID NO: 2 - DNA, 58 bp, synthetic construct
  source 1..58: mol_type = other DNA
actctttccctacacgacgctcttccgatcatcggaagagcacacgtctgaactccag

SEQ ID NO: 3 - DNA, 57 bp, synthetic construct
  source 1..57: mol_type = other DNA
actctttccctacacgacgctcttccgatatcggaagagcacacgtctgaactccag

SEQ ID NO: 4 - DNA, 55 bp, synthetic construct
  source 1..55: mol_type = other DNA
actctttccctacacgacgctcttccgatcggaagagcacacgtctgaactccag

SEQ ID NO: 5 - DNA, 60 bp, synthetic construct
  source 1..60: mol_type = other DNA
actctttccctacacgacgctcttccgatcagatcggaagagcacacgtctgaactccag

SEQ ID NO: 6 - DNA, 60 bp, synthetic construct
  source 1..60: mol_type = other DNA
actctttccctacacgacgctcttccgatctgatcggaagagcacacgtctgaactccag

SEQ ID NO: 7 - DNA, 60 bp, synthetic construct
  source 1..60: mol_type = other DNA
actctttccctacacgacgctcttccgatcggatcggaagagcacacgtctgaactccag

SEQ ID NO: 8 - DNA, 60 bp, synthetic construct
  source 1..60: mol_type = other DNA
actctttccctacacgacgctcttccgatccgatcggaagagcacacgtctgaactccag

SEQ ID NO: 9 - DNA, 60 bp, synthetic construct
  source 1..60: mol_type = other DNA
tgagaaagggatgtgctgcgagaaggctagactagccttctcgtgtgcagacttgaggtc

SEQ ID NO: 10 - DNA, 60 bp, synthetic construct
  source 1..60: mol_type = other DNA
tgagaaagggatgtgctgcgagaaggctagtctagccttctcgtgtgcagacttgaggtc

SEQ ID NO: 11 - DNA, 60 bp, synthetic construct
  source 1..60: mol_type = other DNA
tgagaaagggatgtgctgcgagaaggctaggctagccttctcgtgtgcagacttgaggtc

SEQ ID NO: 12 - DNA, 60 bp, synthetic construct
  source 1..60: mol_type = other DNA
tgagaaagggatgtgctgcgagaaggctagcctagccttctcgtgtgcagacttgaggtc

SEQ ID NO: 13 - DNA, 47 bp, synthetic construct
  source 1..47: mol_type = other DNA
ttaactttaagaaggagatataaccatgaaaatcgaagaaggtaaag

SEQ ID NO: 14 - DNA, 47 bp, synthetic construct
  source 1..47: mol_type = other DNA
ttaactttaagaaggagatatacccatgaaaatcgaagaaggtaaag

SEQ ID NO: 15 - DNA, 47 bp, synthetic construct
  source 1..47: mol_type = other DNA
ttaactttaagaaggagatatagccatgaaaatcgaagaaggtaaag

SEQ ID NO: 16 - DNA, 47 bp, synthetic construct
  source 1..47: mol_type = other DNA
ttaactttaagaaggagatatatccatgaaaatcgaagaaggtaaag

SEQ ID NO: 17 - DNA, 48 bp, synthetic construct
  source 1..48: mol_type = other DNA
ttaactttaagaaggagatataaaccatgaaaatcgaagaaggtaaag

SEQ ID NO: 18 - DNA, 48 bp, synthetic construct
  source 1..48: mol_type = other DNA
ttaactttaagaaggagatatacaccatgaaaatcgaagaaggtaaag

SEQ ID NO: 19 - DNA, 48 bp, synthetic construct
  source 1..48: mol_type = other DNA
ttaactttaagaaggagatatagaccatgaaaatcgaagaaggtaaag

SEQ ID NO: 20 - DNA, 48 bp, synthetic construct
  source 1..48: mol_type = other DNA
ttaactttaagaaggagatatataccatgaaaatcgaagaaggtaaag

SEQ ID NO: 21 - DNA, 49 bp, synthetic construct
  source 1..49: mol_type = other DNA
ttaactttaagaaggagatatatgaccatgaaaatcgaagaaggtaaag

SEQ ID NO: 22 - DNA, 49 bp, synthetic construct
  source 1..49: mol_type = other DNA
ttaactttaagaaggagatataggaccatgaaaatcgaagaaggtaaag

SEQ ID NO: 23 - DNA, 49 bp, synthetic construct
  source 1..49: mol_type = other DNA
ttaactttaagaaggagatatagaaccatgaaaatcgaagaaggtaaag

SEQ ID NO: 24 - DNA, 50 bp, synthetic construct
  source 1..50: mol_type = other DNA
ttaactttaagaaggagatatacctaccatgaaaatcgaagaaggtaaag

SEQ ID NO: 25 - DNA, 50 bp, synthetic construct
  source 1..50: mol_type = other DNA
ttaactttaagaaggagatatatggaccatgaaaatcgaagaaggtaaag

SEQ ID NO: 26 - DNA, 50 bp, synthetic construct
  source 1..50: mol_type = other DNA
ttaactttaagaaggagatataatgaccatgaaaatcgaagaaggtaaag

SEQ ID NO: 27 - DNA, 52 bp, synthetic construct
  source 1..52: mol_type = other DNA
ttaactttaagaaggagatatacaggtaccatgaaaatcgaagaaggtaaag

SEQ ID NO: 28 - DNA, 52 bp, synthetic construct
  source 1..52: mol_type = other DNA
ttaactttaagaaggagatatatgttgaccatgaaaatcgaagaaggtaaag

SEQ ID NO: 29 - DNA, 57 bp, synthetic construct
  source 1..57: mol_type = other DNA
ttaactttaagaaggagatataccccggtctcaccatgaaaatcgaagaaggtaaag

SEQ ID NO: 30 - DNA, 57 bp, synthetic construct
  source 1..57: mol_type = other DNA
ttaactttaagaaggagatatactctggctacaccatgaaaatcgaagaaggtaaag

SEQ ID NO: 31 - protein, 167 aa, synthetic construct
MHHHHHHGSGMLLTGKLYKEEKQKFYDAQNGKCLICQRELNPDVQANHLDHDHELNGPKAGKVRGLLCNLCNAAEGQMKHKFNRSGLKGQGVDYLEWLENLLTYLKSDYTQNNIHPNFVGDKSKEFSRLGKEEMMAEMLQRGFEYNESDTKTQLIASFKKQLRKSLK

SEQ ID NO: 32 - protein, 267 aa, synthetic construct
MMKYKILENPNCEEAYDLIEEGLRKKATITIFACCKVEYEGRALSQLNWGERIILIKPDGSFLIHQEKKVEPVNWQPPKSRTRSYIKDNKLILESHRRTPKELLEVEIRKIQFINYANIEDYEELEQAGYEKDMGDMIMEKPHMIEEGFKPTAREYSVEHGFIDILGKDKDGNLMVLELKARKAGVSAVKQIRRYLTDFENTENDYLKECGVQKKKIRGLLVAPSIGEDAKELIEEEGIEFVSVEPPKELKRDKKVTLDAFHHHHHH

SEQ ID NO: 33 - DNA, 37 bp, synthetic construct
  source 1..37: mol_type = other DNA
atgggtgaaccagcgttcacctctttcccgtctctcc

SEQ ID NO: 34 - DNA, 59 bp, synthetic construct
  source 1..59: mol_type = other DNA
gcccacggcatcatgttacgtttcagtttacccagaaccgggagagacgggaaagaggt

SEQ ID NO: 35 - DNA, 59 bp, synthetic construct
  source 1..59: mol_type = other DNA
gtaacatgatgccgtgggcgctgcagaaaaaacgtgaaatccacatggccaaagcgcac

SEQ ID NO: 36 - DNA, 59 bp, synthetic construct
  source 1..59: mol_type = other DNA
agtcagacgcatcggcagcgcagaacgggcagcacggcgacggtgcgctttggccatgt

SEQ ID NO: 37 - DNA, 59 bp, synthetic construct
  source 1..59: mol_type = other DNA
ctgccgatgcgtctgacttcttgcatcttccgtcgtcctgttacccgtatccgttctca

SEQ ID NO: 38 - DNA, 56 bp, synthetic construct
  source 1..56: mol_type = other DNA
gctcgtcacctttacgacgacgaacctggttgtccgggtgagaacggatacgggta

SEQ ID NO: 39 - DNA, 59 bp, synthetic construct
  source 1..59: mol_type = other DNA
cgtcgtaaaggtgacgagcacctggaaaaaccgcagcagctgtgcgcgtatcgccgtct

SEQ ID NO: 40 - DNA, 59 bp, synthetic construct
  source 1..59: mol_type = other DNA
agaagaaccttcaccctgagaagagcatggctgcagtgcctgcagacggcgatacgcgc

SEQ ID NO: 41 - DNA, 59 bp, synthetic construct
  source 1..59: mol_type = other DNA
tctcagggtgaaggttcttctccgctgcacctcgaatctgttctgtctatcctggcacc

SEQ ID NO: 42 - DNA, 59 bp, synthetic construct
  source 1..59: mol_type = other DNA
ccgcacccgcacggtccagagattcacccgcggtacccggtgccaggatagacagaaca

SEQ ID NO: 43 - DNA, 59 bp, synthetic construct
  source 1..59: mol_type = other DNA
ccgtgcgggtgcggaacgtgttcgctccccgctggaacctaccccaggtcgtttcccgg

SEQ ID NO: 44 - DNA, 59 bp, synthetic construct
  source 1..59: mol_type = other DNA
gcagctggcaacccatacccggggtcggaccaccagcaacagccgggaaacgacctggg

SEQ ID NO: 45 - DNA, 59 bp, synthetic construct
  source 1..59: mol_type = other DNA
gtatgggttgccagctgccaccgccgctctctggtcagctggttacccctgcggacatt

SEQ ID NO: 46 - DNA, 59 bp, synthetic construct
  source 1..59: mol_type = other DNA
gacgttcacgcgcctttttaacgcgacgcgcttggcgacgaatgtccgcaggggtaacc

SEQ ID NO: 47 - DNA, 59 bp, synthetic construct
  source 1..59: mol_type = other DNA
aaaaggcgcgtgaacgtctggcgaaggcgctccaggcggaccgcctcgctcgtcgtgca

SEQ ID NO: 48 - DNA, 55 bp, synthetic construct
  source 1..55: mol_type = other DNA
ttaatggtgatgatggtggtgaccgccagtcagcatctctgcacgacgagcgagg

SEQ ID NO: 49 - DNA, 645 bp, synthetic construct
  source 1..645: mol_type = other DNA
atgggtgaaccagcgttcacctctttcccgtctctcccggttctgggtaaactgaaacgtaacatgatgccgtgggcgctgcagaaaaaacgtgaaatccacatggccaaagcgcaccgtcgccgtgctgcccgttctgcgctgccgatgcgtctgacttcttgcatcttccgtcgtcctgttacccgtatccgttctcacccggacaaccaggttcgtcgtcgtaaaggtgacgagcacctggaaaaaccgcagcagctgtgcgcgtatcgccgtctgcaggcactgcagccatgctcttctcagggtgaaggttcttctccgctgcacctcgaatctgttctgtctatcctggcaccgggtaccgcgggtgaatctctggaccgtgcgggtgcggaacgtgttcgctccccgctggaacctaccccaggtcgtttcccggctgttgctggtggtccgaccccgggtatgggttgccagctgccaccgccgctctctggtcagctggttacccctgcggacattcgtcgccaagcgcgtcgcgttaaaaaggcgcgtgaacgtctggcgaaggcgctccaggcggaccgcctcgctcgtcgtgcagagatgctgactggcggtcaccaccatcatcaccattaa

SEQ ID NO: 50 - DNA, 48 bp, synthetic construct
  source 1..48: mol_type = other DNA
ggtcaccaccatcatcaccattaacatatggtgcactctcagtacaat

SEQ ID NO: 51 - DNA, 46 bp, synthetic construct
  source 1..46: mol_type = other DNA
agaggtgaacgctggttcacccatggtgcctaatgagtgagctaac

SEQ ID NO: 52 - DNA, 60 bp, synthetic construct
  source 1..60: mol_type = other DNA
tcagcaagggctgagggcgcccaatacgcaaaccgcctctccccgcgcgttggccgattc

SEQ ID NO: 53 - DNA, 60 bp, synthetic construct
  source 1..60: mol_type = other DNA
aggtttcccgactggaaagcgggcagtgagcgcaacgcaattaatgtgagttagctcact

SEQ ID NO: 54 - DNA, 60 bp, synthetic construct
  source 1..60: mol_type = other DNA
acactttatgcttccggctcgtatgttgtgtggaattgtgagcggataacaatttcacac

SEQ ID NO: 55 - DNA, 60 bp, synthetic construct
  source 1..60: mol_type = other DNA
aaaggtgaagaactgtttacgggcgttgtgccgatcctggtggaactggacggtgatgtt

SEQ ID NO: 56 - DNA, 60 bp, synthetic construct
  source 1..60: mol_type = other DNA
gagtggcgaaggtgaaggcgatgcgacctatggtaaactgacgctgaaatttatttgcac

SEQ ID NO: 57 - DNA, 60 bp, synthetic construct
  source 1..60: mol_type = other DNA
cgtggccgaccctggtcaccaccctgacctacggtgtgcagtgtttcgcacgctatccgg

SEQ ID NO: 58 - DNA, 60 bp, synthetic construct
  source 1..60: mol_type = other DNA
tttttcaaaagcgctatgccggaaggttacgttcaggaacgtaccattttctttaaagat

SEQ ID NO: 59 - DNA, 60 bp, synthetic construct
  source 1..60: mol_type = other DNA
cgccgaagtcaaatttgaaggtgatacgctggtgaaccgtattgaactgaaaggcatcga

SEQ ID NO: 60 - DNA, 60 bp, synthetic construct
  source 1..60: mol_type = other DNA
tcctgggccataaactggaatacaactacaactcacacaaagtttacattaccgcggata

SEQ ID NO: 61 - DNA, 60 bp, synthetic construct
  source 1..60: mol_type = other DNA
gtcaacttcaaaacgcgtcataacatcgaagatggctctgtgcaactggccgaccactac

SEQ ID NO: 62 - DNA, 60 bp, synthetic construct
  source 1..60: mol_type = other DNA
tgatggcccggttctgctgccggacaatcattatctgtccacccagtcagcactgtcgaa

SEQ ID NO: 63 - DNA, 60 bp, synthetic construct
  source 1..60: mol_type = other DNA
accacatggtgctgctggaatttgttaccgcggccggtattacgctgggcatggatgaac

SEQ ID NO: 64 - DNA, 60 bp, synthetic construct
  source 1..60: mol_type = other DNA
tcagcggaagctgaggttatttgtacagttcatccatgcccagcgtaataccggccgcgg

SEQ ID NO: 65 - DNA, 60 bp, synthetic construct
  source 1..60: mol_type = other DNA
ttccagcagcaccatgtggtcgcgtttttcattcggatctttcgacagtgctgactgggt

SEQ ID NO: 66 - DNA, 60 bp, synthetic construct
  source 1..60: mol_type = other DNA
gcagcagaaccgggccatcaccgatcggggtgttttgctggtagtggtcggccagttgca

SEQ ID NO: 67 - DNA, 60 bp, synthetic construct
  source 1..60: mol_type = other DNA
tgacgcgttttgaagttgactttgataccgtttttctgtttatccgcggtaatgtaaact

SEQ ID NO: 68 - DNA, 60 bp, synthetic construct
  source 1..60: mol_type = other DNA
ttccagtttatggcccaggatattaccgtcttctttgaaatcgatgcctttcagttcaat

SEQ ID NO: 69 - DNA, 60 bp, synthetic construct
  source 1..60: mol_type = other DNA
cttcaaatttgacttcggcgcgggttttgtagttgccgtcatctttaaagaaaatggtac

SEQ ID NO: 70 - DNA, 60 bp, synthetic construct
  source 1..60: mol_type = other DNA
ggcatagcgcttttgaaaaagtcgtgttgtttcatatgatccggatagcgtgcgaaacac

SEQ ID NO: 71 - DNA, 60 bp, synthetic construct
  source 1..60: mol_type = other DNA
ggtgaccagggtcggccacggaaccggcagtttaccggtggtgcaaataaatttcagcgt

SEQ ID NO: 72 - DNA, 60 bp, synthetic construct
  source 1..60: mol_type = other DNA
cgccttcaccttcgccactcacagagaatttatgaccattaacatcaccgtccagttcca

SEQ ID NO: 73 - DNA, 60 bp, synthetic construct
  source 1..60: mol_type = other DNA
gtaaacagttcttcacctttgctaaccatagctgtttcctgtgtgaaattgttatccgct

SEQ ID NO: 74 - DNA, 60 bp, synthetic construct
  source 1..60: mol_type = other DNA
gagccggaagcataaagtgtaaagcctggggtgcctaatgagtgagctaactcacattaa

SEQ ID NO: 75 - DNA, 60 bp, synthetic construct; identical to SEQ ID NO: 74
  source 1..60: mol_type = other DNA

SEQ ID NO: 76 - DNA, 40 bp, synthetic construct
  source 1..40: mol_type = other DNA
ataatcagcttccgctgagggtaccgagctcgaattcact

SEQ ID NO: 77 - DNA, 37 bp, synthetic construct
  source 1..37: mol_type = other DNA
gcgccctcagcccttgctgagctcttccgcttcctcg

SEQ ID NO: 78 - DNA, 967 bp, synthetic construct
  source 1..967: mol_type = other DNA
tcagcaagggctgagggcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatggttagcaaaggtgaagaactgtttacgggcgttgtgccgatcctggtggaactggacggtgatgttaatggtcataaattctctgtgagtggcgaaggtgaaggcgatgcgacctatggtaaactgacgctgaaatttatttgcaccaccggtaaactgccggttccgtggccgaccctggtcaccaccctgacctacggtgtgcagtgtttcgcacgctatccggatcatatgaaacaacacgactttttcaaaagcgctatgccggaaggttacgttcaggaacgtaccattttctttaaagatgacggcaactacaaaacccgcgccgaagtcaaatttgaaggtgatacgctggtgaaccgtattgaactgaaaggcatcgatttcaaagaagacggtaatatcctgggccataaactggaatacaactacaactcacacaaagtttacattaccgcggataaacagaaaaacggtatcaaagtcaacttcaaaacgcgtcataacatcgaagatggctctgtgcaactggccgaccactaccagcaaaacaccccgatcggtgatggcccggttctgctgccggacaatcattatctgtccacccagtcagcactgtcgaaagatccgaatgaaaaacgcgaccacatggtgctgctggaatttgttaccgcggccggtattacgctgggcatggatgaactgtacaaataatcagcttccgctgagg

SEQ ID NO: 79 - DNA, 17 bp, synthetic construct
  source 1..17: mol_type = other DNA
tcagcaagggctgaggg

SEQ ID NO: 80 - DNA, 28 bp, synthetic construct
  source 1..28: mol_type = other DNA
cctcagcggaagctgattatttgtacag

SEQ ID NO: 81 - DNA, 10 bp, synthetic construct
  source 1..10: mol_type = other DNA
tatatatata

(19) United States
(12) Patent Application Publication
(10) Pub. No.: US 2025/0027064 A1
(43) Pub. Date: Jan. 23, 2025

(54) Polynucleotide Error Recognition Methods and Compositions

(71) Applicant: New England Biolabs, Inc., Ipswich, MA (US)

(72) Inventors: Pei-Chung Hsieh, Topsfield, MA (US); Andrew F. Gardner, Manchester, MA (US); Ashley Luck, Ipswich, MA (US)

(73) Assignee: New England Biolabs, Inc., Ipswich, MA (US)

(21) Appl. No.: 18/356,052

(22) Filed: Jul. 20, 2023

(51) Int. Cl. C12N 9/22; C12Q 1/6806; C12Q 1/686

(52) U.S. Cl. CPC C12N 9/22; C12Q 1/6806; C12Q 1/686

(57) ABSTRACT

The present disclosure relates, according to some embodiments, to systems, apparatus, compositions, kits, workflows, and/or methods that address errors in synthesizing and/or copying polynucleotide sequences. The present disclosure provides, for example, systems, apparatus, compositions, kits, workflows, and methods for detecting, reducing, and/or removing sequence errors including mismatches and/or indels. Enzyme composition may include, in some embodiments, a T4EndoVII endonuclease and a mismatch endonuclease, wherein the composition is cell-free, the composition has a temperature of 30° C. to 45° C., the composition comprises ≥1 unit of T4EndoVII per μL and ≥1 unit of μL of the mismatch endonuclease, the composition comprises ≥0.2 ng T4EndoVII per 20 μL and ≥7 ng mismatch endonuclease per 20 μL, the composition further comprises a buffering agent, and/or the mismatch endonuclease is EndoMS.

A Sequence Listing is provided herewith as a Sequence Listing XML, “NEB-460.xml” created on Jul. 20, 2023, and having a size of 74.2 KB. This Sequence Listing is incorporated herein in its entirety by this reference.

### BACKGROUND

The power of and applications for synthetic biology continue to grow. Yet, production of a homogeneous population of polynucleotides having the desired sequence remains a challenge. For example, chemical synthesis of oligonucleotides may include unwanted side reactions that result in a fraction of the resulting population of oligonucleotide strands having a nucleotide sequence that differs from the desired sequence. If left unchecked, subsequent assembly and/or amplification using oligonucleotides containing the undesired sequence(s) may result in a heterogeneous population of duplex polynucleotides, with some members having the desired sequence, but others having one or more alternate sequences. Depending on how prevalent errors are in the oligonucleotide and/or the conditions of assembly/amplification, the final population may have few or no copies of the desired full-length sequence.

### SUMMARY

Accordingly, needs have arisen for improved systems, apparatus, compositions, kits, workflows, and/or methods that address errors in synthesizing and/or copying polynucleotide sequences. The present disclosure relates to such systems, apparatus, compositions, kits, workflows, and methods. For example, systems, apparatus, compositions, kits, workflows, and methods for detecting, reducing, and/or removing sequence errors including mismatches and/or indels are provided.

The present disclosure relates, in some embodiments, to an endonuclease composition. For example, an enzyme composition may comprise a T4EndoVII endonuclease and a mismatch endonuclease, wherein (a) the composition is cell-free, or (b) the composition has a temperature of 30° C. to 45° C., or (c) the composition comprises ≥1 unit of T4EndoVII per μL and >1 unit of the mismatch endonuclease per μL, or (d) the composition comprises ≥0.2 ng T4EndoVII per 20 μL and ≥7 ng mismatch endonuclease per 20 μL, or (e) the composition further comprises a buffering agent, or (f) the mismatch endonuclease is EndoMS or any combination thereof. An enzyme composition may further comprise, in some embodiments, one or more additional components (e.g., enzymes, additives, substrates). For example, an enzyme composition may further comprise a polymerase, a base editor, a restriction enzyme, NTPs, or any combinations thereof. Enzyme compositions may have any desired form including, for example, a dried form, a freeze dried form, a lyophilized form, a crystalline form, an aqueous form, a liquid form, or an immobilized form. In some embodiments, an enzyme composition may comprise dsDNA molecules comprising (a) heteroduplex dsDNA comprising at least one error (e.g., a mismatch or an indel) and (b) homoduplex dsDNA. Homoduplex dsDNA may be identical to a reference DNA in length and sequence. For clarity, homoduplex DNA may include an error with respect to a reference sequence, but the strands of the homoduplex DNA are complementary such that the homoduplex itself is free of mismatches and indels. Homoduplex DNA optionally may have blunt ends or may comprise short (e.g., 1-6 nt) 5′ and/or 3′ overhangs. In some embodiments, dsDNA molecules may comprise heteroduplex dsDNA substrates comprising different errors. For example, dsDNA molecules may comprise a first species of heteroduplex dsDNA and a second species of heteroduplex dsDNA wherein the at least one error of the first species differs from the at least one error of the second species. In some embodiments, heteroduplex dsDNA comprises both mismatches and indels.

The present disclosure relates to kits, in some embodiments. A kit may comprise, for example, (a) a T4EndoVII endonuclease, (b) a mismatch endonuclease (e.g., EndoMS), (c) and/or (c) a buffer or buffering agent, an additive, instructions for use, or any combinations thereof. A T4EndoVII endonuclease and a mismatch endonuclease of a kit may be included together in a single composition, for example, as described above and throughout this disclosure. Any kit component up to the entire kit may have may have any desired form (e.g., a liquid form, a lyophilized form, a dried form).

The present disclosure further relates to methods for DNA authentication (e.g., detecting and/or correcting errors in a dsDNA population). A method of DNA authentication may include, for example, contacting (a) dsDNA molecules comprising (i) heteroduplex dsDNA comprising at least one error (e.g., a mismatch or an indel) and (ii) homoduplex dsDNA, (b) a T4EndoVII endonuclease, and (c) a mismatch endonuclease (e.g., EndoMS) to form authentication products comprising uncut dsDNA molecules and/or dsDNA fragments corresponding to cleavage of heteroduplex dsDNA at the error(s). A method may include forming dsDNA molecules (e.g., prior to contacting with T4EndoVII endonuclease and mismatch endonuclease), for example, by denaturing and reannealing a source dsDNA to produce the dsDNA molecules. A T4EndoVII endonuclease and a mismatch endonuclease may be provided for use in a method in any desired form, for example, as individual components to be combined, as a mixture, or as a kit, in each case, as such components, mixtures, and kits are described above and throughout this disclosure.

In some embodiments, a homoduplex dsDNA may be error-free in that each strand of the homoduplex dsDNA matches a reference sequence or its complement. Endonuclease compositions and methods, in some embodiments, may have little to no ability to cleave homoduplex dsDNA. For example, combinations of T4EndoVII and mismatch endonuclease may cleave the homoduplex error-free dsDNA fragment at a rate ≤80%, ≤70%, ≤60%, ≤ 50%, ≤40%, ≤30%, ≤20%, ≤10%, ≤5%, ≤3%, or ≤1% (in each case mole % of homoduplex molecules cut to total homoduplex molecules). Authentication products including cleaved dsDNA substrates may correspond in length (+5 nucleotides or less) to the number of nucleotides from (a) a 5′ end of the dsDNA substrate to the error, (b) from one error to an adjacent error, or (c) from the error to a 3′ end of the dsDNA substrate. Source dsDNA, according to some embodiments, may comprise PCR amplicons from a base-edited genome or a chemically synthesized oligonucleotide, or DNA purified from a phage or cell. Authentication products may comprise less heteroduplex dsDNA than the starting population of dsDNA molecules. For example, authentication products may be free of heteroduplex dsDNA or may comprise ≤90%, ≤80%, ≤70%, ≤60%, ≤50%, ≤40%, ≤30%, ≤20%, ≤10%, ≤5%, ≤3%, or ≤1% (in each case mole %) of the heteroduplex dsDNA in the dsDNA molecules prior to contact with endonucleases.

The present disclosure relates, in some embodiments, to methods of screening and forming screening products. For example, a method of forming screening products may include (a) performing DNA authentication as disclosed above and throughout this specification to form authentication products comprising uncut dsDNA molecules and/or dsDNA fragments, (b) amplifying (e.g., PCR amplifying) the authentication products to form amplification products (e.g., comprising one or more copies of the dsDNA molecules and/or one or more copies of the dsDNA fragments), (c) transforming bacteria with the amplification products to form transformation products (e.g., including transformed bacterial cells comprising at least one amplification product), and/or (d) screening the transformation products to form screening products (e.g., bacterial cells comprising at least one copy of one of the starting dsDNA molecules). In some embodiments, amplifying the authentication products comprises PCR amplifying in a mixture comprising a polymerase, nucleotide triphosphates (NTPs), and primers (e.g., primers configured to hybridize with at least a portion of one or more dsDNA fragments). A method may comprise, in some embodiments, forming the amplification products and amplifying the amplification products in the same mixture, the mixture comprising the T4EndoVII, the mismatch endonuclease, the polymerase, the NTPs, and the primers. According to some embodiments, amplification products of authentication products have fewer errors than amplification products arising from the starting dsDNA molecules not subjected to authentication. For example, amplification products of authentication products may have ≤90%, ≤80%, ≤70%, ≤60%, ≤50%, ≤40%, ≤30%, ≤20%, ≤10%, ≤5%, ≤3%, or ≤1% as many errors as a corresponding quantity of amplification products arising from the same starting dsDNA not authenticated (e.g., not contacted with the T4EndoVII or the mismatch endonuclease). Transformation may comprise, in some embodiments, assembling the dsDNA fragments into a vector and transforming a competent bacteria with the vector. In some embodiments, screening transformation products may comprise plating the bacteria on agar and selecting individual colonies, which form the screening products.

In some embodiments, the screening products comprise one or more error-free clones of the starting dsDNA molecules. The same starting dsDNA molecules may be amplified with and without authentication (e.g., without contact with the T4EndoVII or the mismatch endonuclease) to separately produce authenticated and non-authenticated amplification products which may be transformed into bacteria to separately produce authenticated and non-authenticated transformation products. These products, in turn, may be screened and the products of each screen may be compared. In some embodiments, the number of correct (e.g., error-free) clones in the screening products of authenticated dsDNA molecules may be ≥1%, ≥2%, ≥5%, ≥7%, ≥10%, ≥25%, ≥50%, ≥75%, ≥100%, ≥125%, ≥150%, ≥175%, or ≥200% more than the number of correct (e.g., error-free) clones in the screening products of non-authenticated dsDNA molecules. Screening products arising from authenticated dsDNA molecules may include correct (e.g., error-free) clones and incorrect (e.g., comprising one or more errors) clones. Authentication screening products may have a fraction of correct clones to total clones (e.g., the sum of the correct and incorrect clones) that is higher (e.g., ≥1.1×, ≥1.2×, ≥1.4×, ≥1.6×, ≥1.8×, ≥2.0×, or ≥2.5× higher) than the fraction of correct clones to total clones from non-authenticated screening products.

The present disclosure also relates to methods of DNA fragment analysis. A method of DNA fragment analysis may include, according to some embodiments. (a) performing DNA authentication as disclosed above and throughout this specification to form authentication products comprising uncut dsDNA molecules and/or dsDNA fragments. (b) analyzing the authentication products to determine the amount of cleaved dsDNA substrates in the authentication products and the amount of uncleaved dsDNA substrates in the authentication products; and (c) determining the proportion of heteroduplex dsDNA in the authentication products, wherein the proportion of heteroduplex dsDNA equals the amount cleaved dsDNA substrates divided by the amount of cleaved dsDNA substrates plus the amount of uncleaved dsDNA substrates. Analyzing may comprise, for example, analyzing the authentication products by gel electrophoresis or microfluidics electrophoresis (e.g., using a Bioanalyzer (Agilent Technologies, Inc.)). Analyzing may comprise, for example, determining the moles of uncleaved dsDNA and the moles of cleaved dsDNA. In some embodiments, the proportion of heteroduplex dsDNA molecules to total dsDNA molecules included in the starting dsDNA molecules equals the proportion of errors in the source dsDNA.

### BRIEF DESCRIPTION OF THE FIGURES

FIG. 1A shows representations of example indels and mismatches in heteroduplex DNA 110 that may be subject to authentication according to embodiments of the present disclosure. Heteroduplex DNA 110 comprises subject strand 111 and reference strand 112, shown as the upper and lower strands, respectively. Subject strand 111 comprises purine: purine (R:R) mismatch 121, insertion 122, deletion 123, pyrimidine: pyrimidine (Y:Y) mismatch 124, and purine:pyrimidine (R:Y) mismatch 125. In each mismatch pair, the first letter indicates the base of subject strand 111 and the second letter indicates the base of reference strand 112. It will be appreciated that mismatch 125 could alternatively be a pyrimidine:purine (Y:R) mismatch in which the base of subject strand 111 is the pyrimidine base. The bases of the indels and mismatches of strand 111 are errors since they do not match the bases of reference strand 112.

FIG. 1B shows representations of example insertions in heteroduplex DNA 130 30) that may be subject to authentication according to embodiments of the present disclosure. Heteroduplex DNA 130 comprises subject strand 131 and reference strand 132, shown as the upper and lower strands, respectively. Upper strand 131 comprises 1 bp insertion 142; 2 bp insertion 143, 3 bp insertion 144, and 5 bp insertion 145. As illustrated, insertions may be viewed as bumps or loops off the otherwise double-stranded structure. Insertions 142, 143, 144, and 145 of subject strand 131 are errors relative to reference strand 132.

FIG. 1C shows representations of example mismatches in heteroduplex DNA 150 that may be subject to authentication according to embodiments of the present disclosure. Heteroduplex DNA 150 comprises subject strand 151 and reference strand 152, shown as the upper and lower strands. Upper strand 151 comprises 1 bp mismatch 162; 2 bp mismatch 163, 3 bp mismatch 164, and 5 bp mismatch 165. Although nucleotides represented as “N” in the figure, it will be understood that Watson-Crick pairs are excluded since these represent mismatches. As illustrated, mismatches may be viewed as bulges in the otherwise double-stranded structure. Mismatches 162, 163, 164, and 165 of subject strand 151 are errors.

FIG. 2A shows a representation of cleavage activity of T4 endonuclease VII with a Holliday junction. The cleavage site includes 4 repeats of sequence ATATATATAT (SEQ ID NO:81) that form 2 hairpins.

FIG. 2B shows a representation of cleavage activity of mismatch endonuclease I with substrates having mismatches shown on the left and cleavage products for each substrate shown on the right.

FIG. 3 is a flow chart representation of example method 300 for authenticating dsDNA 320, according to some embodiments of the present disclosure.

FIG. 4 illustrates example method 400 for reducing or eliminating authentication-resistant duplexes in authentication substrates. Examples 5 and 6 use an embodiment of method 400 to prepare a 672 bp homogeneous heteroduplex substrates for use in evaluating the cleavage specificity of an endonuclease composition comprising T4 Endo VII and Mismatch Endo I.

FIG. 5 illustrates example method 500 for assessing the prevalence of molecules comprising one or more errors in a dsDNA population including preparing heterogenous heteroduplex substrates.

FIG. 6A and FIG. 6B illustrate example populations of dsDNA that may be assayed and/or authenticated in accordance with embodiments of the disclosure.

FIG. 7 is a schematic representation of example method 700 for authenticating dsDNA 720, according to some embodiments of the present disclosure.

FIG. 8 illustrates example results with 645 bp DNA assembly products that were sequenced after assembly (top set), authenticated using an example composition comprising T4EndoVII and EndoMS of the present disclosure (middle set), or treated with CORRECTASE™ (Thermo Fisher. US) in accordance with the manufacturer's instructions (bottom set), wherein red dots indicate an indel error and blue dots indicate a mismatch.

FIG. 9A shows agarose gels of example authentication products obtained by contacting 60-mers with the indicated errors with T4EndoVII/EndoMS. FIG. 9B shows agarose gels of products obtained by contacting the same 60-mers of with commercial T7 Endonuclease I.

FIG. 10A and FIG. 10B show agarose gels of example authentication products obtained by contacting 60-mer oligonucleotides with the indicated mismatches or indels. FIG. 10A shows results with a two-enzyme combination (T4EndoVII+EndoMS) and FIG. 10B shows results with mismatch endonuclease I alone or T7 endonuclease I alone.

FIG. 11A shows agarose gels of example cleavage products obtained by contacting homogeneous heteroduplex dsDNA substrates (672-mers) with the indicated 1 bp mismatch errors with T4 endonuclease VII (“T4Endo7”) and substrates prepared according to method 400. The schematic to the left indicates the location of uncut substrate (“S”), a larger product (“P1”) and a smaller product (“P2”). FIG. 11B shows agarose gels of example cleavage products obtained by contacting 672-mers with the 1 bp mismatch errors indicated in FIG. 11A with mismatch endonuclease (“EndoMS”). The schematic to the left indicates the location of uncut substrate (“S”), a larger product (“P1”) and a smaller product (“P2”). FIG. 11C shows agarose gels of example authentication cleavage products obtained by contacting 672-mers with the 1 bp mismatch or indel errors indicated in FIG. 11A with T4 endonuclease VII and mismatch endonuclease (“T4Endo7 & EndoMS”). The schematic to the left indicates the location of uncut substrate (“S”), a larger product (“P1”) and a smaller product (“P2”). FIG. 11D shows agarose gels for a parallel experiment using T7 endonuclease I. FIG. 11E shows agarose gels for a parallel experiment using CorrectASE. As illustrated. T4 endonuclease VII and mismatch endonuclease together efficiently cleaved all of the tested mismatches.

FIG. 12 shows agarose gels of example authentication products obtained by contacting heterogeneous heteroduplex dsDNA substrates (around 672 mer) with the indicated mismatches or indels with a two-enzyme combination (T4EndoVII+EndoMS), mismatch endonuclease I alone or T7 endonuclease I alone.

FIG. 13 shows a comparison between theoretical and example experimental DNA fragment analysis. Heteroduplex DNA substrates are prepared as described using using method 500 illustrated in FIG. 5. Mismatch cleavage analyses are compared using endonuclease compositions either comprising T7 endonuclease I alone or comprising T4EndoVII and EndoMS. Different types of initial substrates with 25% of mutation population are set up as indicated in the X-axis followed by the digestion of enzyme. For example, A/C, G/T refer to mismatches in the initial substrate pool; 2 bp mismatch means there are 25% of initial substrate with 2 bp deletion. Methods of calculating mutation population (% modification) are shown in Example 2. While the endonuclease composition comprising T7 endonuclease I alone appears to perform as well as the composition comprising T4EndoVII and EndoMS where the mismatch or indel is more than 3 bp, the endonuclease composition comprising T4EndoVII and EndoMS performs better than the composition comprising T7 endonuclease I alone where the errors are 1-2 bp mismatches.

### BRIEF DESCRIPTION OF THE SEQUENCES

Some embodiments of this disclosure relate to the following provided sequences of example polynucleotides and/or example polypeptides.

SEQ ID NOS: 1-12 are example 60-mer oligos used to create example heteroduplex DNA molecules with one nucleotide mismatches or one-, two-, three- or five-base indels.

SEQ ID NOS: 13-30 represent the sequence of the central region of a series of example substrates for authentication to highlight the portion of each that includes a mismatch or indel error. The sequence of the respective molecules outside the central regions shown are error-free.

SEQ ID NO: 31 is an example sequence of a T4EndoVII having an N-terminal polyhistidine tag.

SEQ ID NO: 32 is an example sequence of a EndoMS having an C-terminal polyhistidine tag.

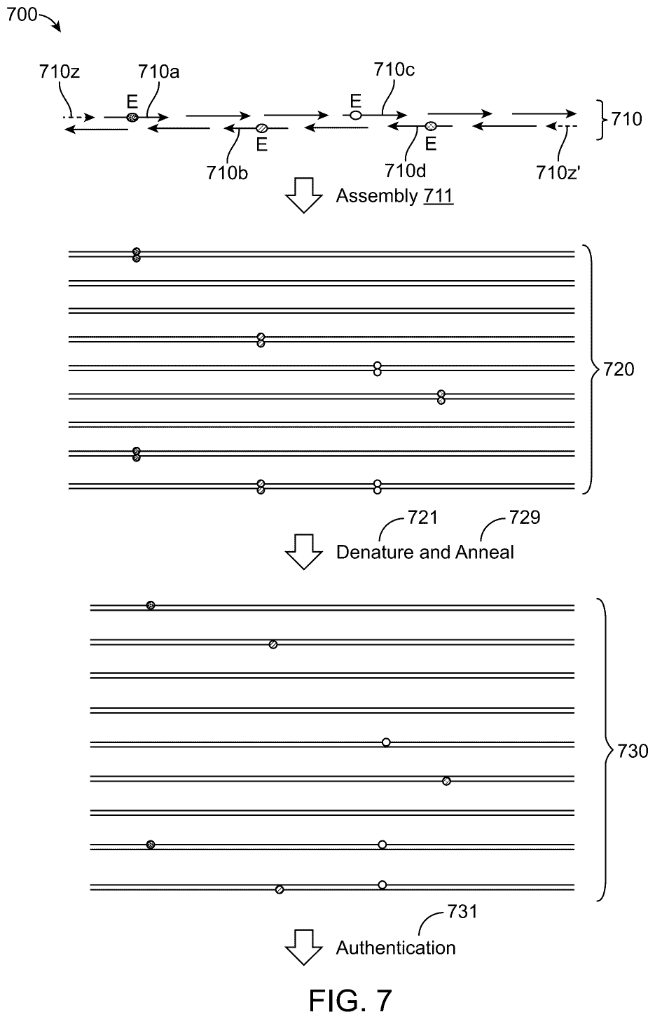

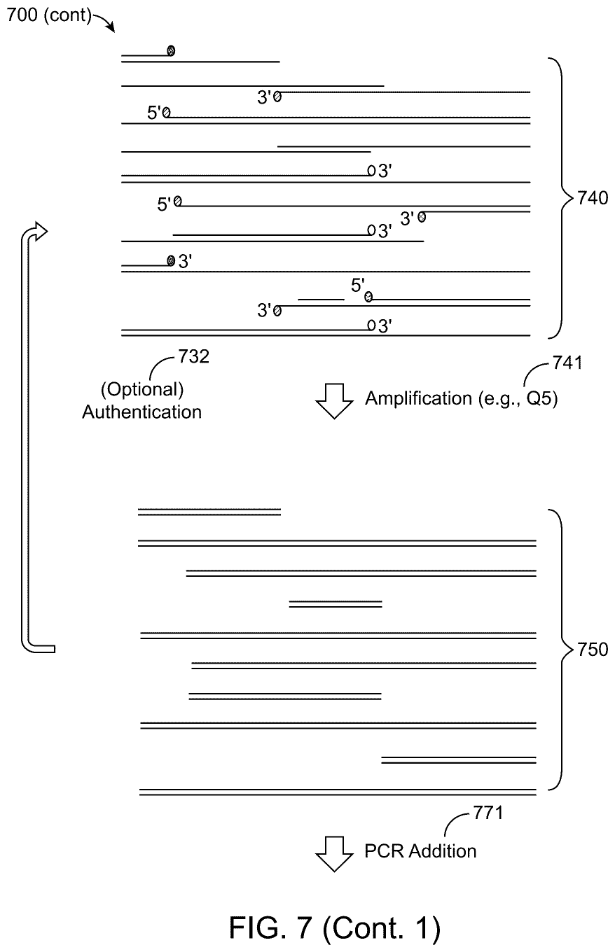

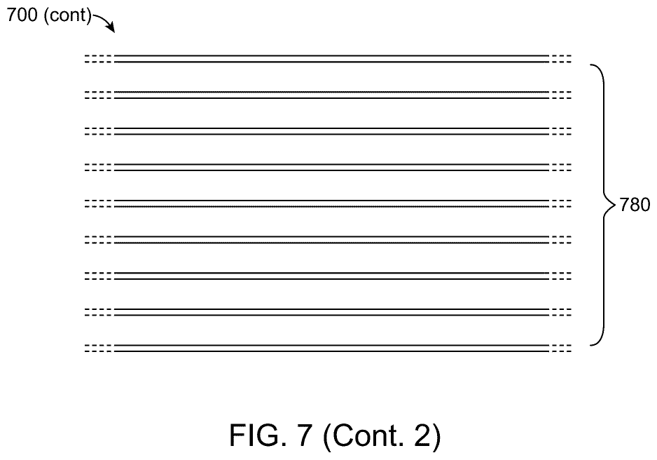

SEQ ID NOS: 33-48 are example oligos that may be used in an assembly process to form an example maltose binding domain. These sequences are examples of oligonucleotide fragments 710 illustrated in FIG. 7.

SEQ ID NO: 49 is an example DNA sequence encoding a maltose binding domain that may be assembled, for example, from SEQ ID NOS: 33-48.

SEQ ID NO: 50 is an example sequence for a forward primer that may be used to amplify a maltose binding domain sequence.

SEQ ID NO: 51 is an example sequence for a reverse primer that may be used to amplify a maltose binding domain sequence.

SEQ ID NOS: 52-75 are example oligos that may be used in an assembly process to form an example green fluorescent protein. These sequences are examples of oligonucleotide fragments 710 illustrated in FIG. 7.

SEQ ID NO: 76 is an example sequence for a forward primer that may be used to amplify an ozGFP_pUC19 sequence.

SEQ ID NO: 77 is an example sequence for a reverse primer that may be used to amplify an ozGFP_pUC19 sequence.

SEQ ID NO: 78 is an example DNA sequence encoding a lacZ plus GFP that may be assembled, for example, from SEQ ID NOS: 52-75.

SEQ ID NO: 79 is an example sequence for a forward primer that may be used to amplify a lacZ-GFP sequence.

SEQ ID NO: 80 is an example sequence for a reverse primer that may be used to amplify a lacZ-GFP sequence.

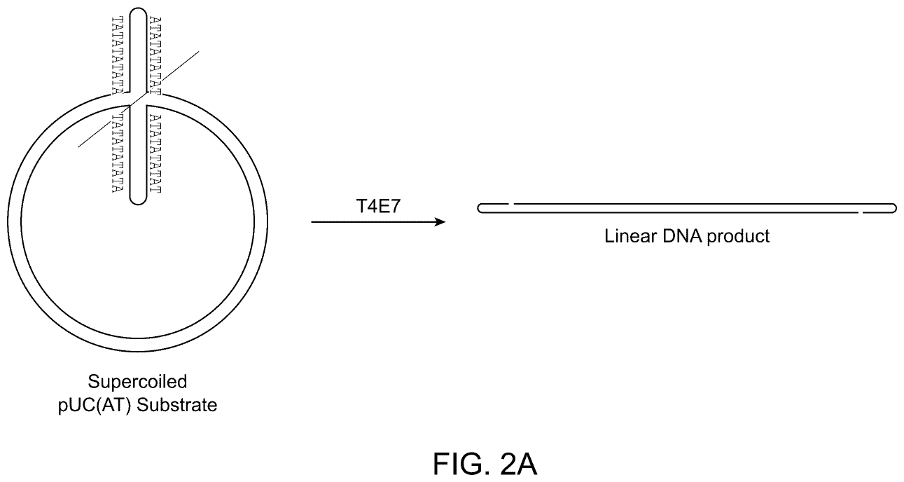

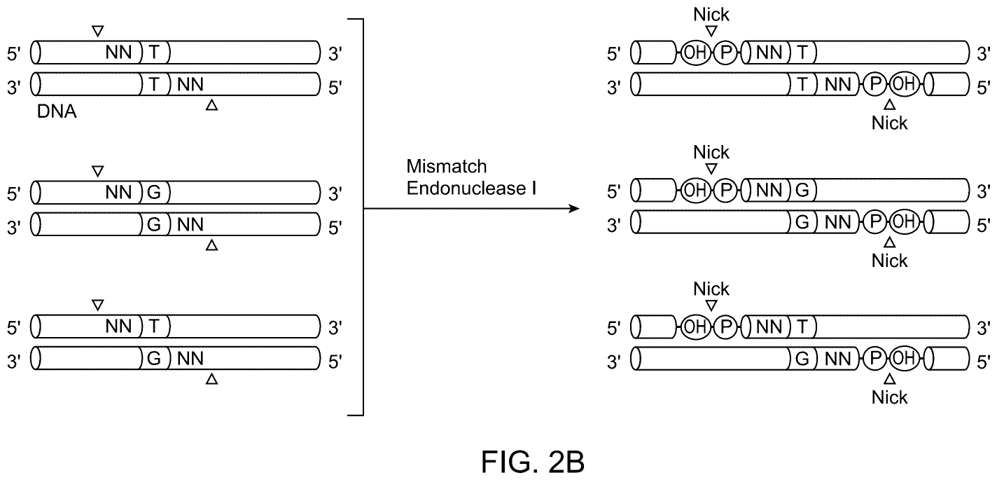

SEQ ID NO: 81 is included in the cut site of T4EndoVII as illustrated in FIG. 2A.

### DETAILED DESCRIPTION

The present disclosure relates to compositions, methods, workflows, and systems for altering the sequence of a subject polynucleotide to better conform (e.g., substantially conform, fully conform) to the sequence of a reference polynucleotide. For example, the present disclosure provides compositions and methods for correcting errors in the sequence of a first polynucleotide relative to the sequence of a reference polynucleotide. Compositions may include, according to some embodiments, at least two endonucleases that cut (e.g., nick) heteroduplex polynucleotides comprising one or more errors. For example, compositions may include T4EndoVII and EndoMS. In some embodiments, a composition may be a cell-free composition, may include either or both endonucleases in a concentration greater than that found in a unmodified wildtype cell, may be lyophilized or otherwise not an aqueous composition. In some embodiments, a composition may contain other enzymes, such as a polymerase or other nucleic acid amplification enzymes, base editors, or restriction enzymes, DNA fragments, error-free DNA fragments, DNA substrates, or any combinations thereof.

In some embodiments, the present disclosure relates to methods of recognizing sequence inconsistencies (e.g., errors) in DNA substrates and cleaving the DNA substrates at the locations of the sequence inconsistencies (e.g., errors) using at least two endonucleases (e.g., T4EndoVII and EndoMS) to form sequence-conformed (e.g., error-corrected) DNA fragments. In some embodiments, recognition and cleavage may occur before or after amplification of DNA fragments. In some embodiments, recognition and cleavage may be part of a method of calculating mutation rates in a population of DNA fragments.

Compositions and methods of the disclosure may create a double-stranded break in a DNA substrate around the inconsistencies (e.g., errors). In some embodiments, double-stranded breaks may result in overhangs. In some embodiments, compositions and methods may further include filling in overhangs, for example, with a polymerase and nucleotide triphosphates (NTPs), resulting in double-stranded DNA without overhangs.

Compositions and methods of the present disclosure, according to some embodiments, may entirely remove DNA fragments with inconsistencies (e.g., errors), resulting in a homogeneous population of DNA molecules, free of sequence inconsistencies. In some embodiments, compositions and methods of the present disclosure may reduce the abundance of DNA fragments with inconsistencies (e.g., errors), resulting in a population of DNA molecules that is substantially homogeneous, for example, wherein ≥80%, ≥82%, ≥85%, ≥ 88%, ≥90%, ≥91%, ≥92%, ≥93%, ≥94%, ≥95%, ≥96%, ≥97%, ≥98%, or ≥99% (in each case mole %) of the molecules in the population have sequence identity.

Compositions and methods of the present disclosure may reduce or entirely remove DNA fragments with errors, resulting in error-corrected DNA fragments, which may contain, in some embodiments, at least 90%, error-free DNA fragments.

Sequence-conformed DNA fragments may be used for subsequent amplification or gene assembly and will result in a greater proportion of assembled genes having the correct DNA sequence than if subsequent amplification or gene assembly were performed on DNA fragments not treated with a combination of endonucleases (e.g., T4EndoVII and EndoMS).

The present disclosure further provides kits containing the compositions or for carrying out the methods.

### General Considerations

Aspects of the present disclosure can be understood in light of the provided descriptions, figures, sequences, embodiments, section headings, and examples, none of which should be construed as limiting the entire scope of the present disclosure in any way. Accordingly, the innovations set forth herein should be construed in view of the full breadth and spirit of the disclosure.

Each of the individual embodiments described and illustrated herein has discrete components and features which can be readily separated from or combined with the components and/or features of any of the other several embodiments without departing from the scope or spirit of the present teachings. Lists of example species within a particular genus may vary in length at different places throughout the disclosure. Species lists shortened for convenience shall not be construed to exclude example species listed elsewhere in the specification. Any recited method can be carried out in the order of events recited or in any other order which is logically possible. Unless otherwise expressly stated to be required herein, each component, feature, and method step disclosed herein is optional and the disclosure contemplates embodiments in which each optional element may be expressly excluded. As such, this statement is intended to serve as antecedent basis for use of such exclusive terminology as “solely,” “only” and the like in connection with the recitation of claim elements or use of a “negative” limitation. It is further intended to serve as antecedent basis for use of such elective terminology as “optionally” and the like in connection with the recitation of one or more claim elements.

Unless otherwise defined, all technical and scientific terms used herein have the same meaning as commonly understood by one of ordinary skill in the art to which this disclosure belongs. Still, certain terms are defined herein with respect to embodiments of the disclosure and for the sake of clarity and ease of reference.

Sources of commonly understood terms and symbols may include: standard treatises and texts such as Kornberg and Baker, DNA Replication, Second Edition (W. H. Freeman, New York, 1992); Lehninger, Biochemistry, Second Edition (Worth Publishers, New York, 1975); Strachan and Read, Human Molecular Genetics, Second Edition (Wiley-Liss, New York, 1999); Eckstein, editor, Oligonucleotides and Analogs: A Practical Approach (Oxford University Press, New York, 1991); Gait, editor, Oligonucleotide Synthesis: A Practical Approach (IRL Press, Oxford, 1984); Singleton, et al., Dictionary of Microbiology and Molecular biology, 2d ed., John Wiley and Sons, New York (1994), and Hale & Markham, the Harper Collins Dictionary of Biology, Harper Perennial, N.Y. (1991) and the like.

As used herein and in the appended claims, the singular forms “a” and “an” include plural referents. For example, the term “a protein” refers to one or more proteins, i.e., a single protein and multiple proteins.

Numeric ranges are inclusive of the numbers defining the range. All numbers should be understood to encompass the midpoint of the integer above and below the integer i.e., the number 2 encompasses 1.5-2.5. The number 2.5 encompasses 2.45-2.55 etc. When sample numerical values are provided, each alone may represent an intermediate value in a range of values and together may represent the extremes of a range unless specified. Ranges (including percent ranges) with only one end point (e.g., ≥90) or ≤10) optionally include a second endpoint 10% higher or 10% lower than the provided endpoint (e.g., ≥90 includes a range of 90-99 and ≤10 includes a range of 1-10). Percent ranges with only one end point (e.g., ≥90% or ≤10%) optionally include a second endpoint at the maximum or minimum percentage (e.g., ≥90% includes a range of 90%-100% and ≤10% includes a range of 0%-10%).

Disclosed compositions, methods, workflows, and systems for altering the sequence of a subject polynucleotide to better conform (e.g., substantially conform, fully conform) to the sequence of a reference polynucleotide may be elaborated in terms of one type of polynucleotide (e.g., DNA). Unless expressly stated otherwise, embodiments with other polynucleotides (e.g., RNA) may be contemplated.

In the context of the present disclosure, “authenticate” refers to any provided means for identifying and/or correcting errors in polynucleotide compositions. Authenticating includes, for example, increasing the homogeneity (e.g., size and/or sequence homogeneity) of the population and/or decreasing heterogeneity (e.g., size and/or sequence heterogeneity) of the population. For example, authenticating includes increasing the fraction of a population of polynucleotides (e.g., dsDNA molecules) having a desired size and/or sequence and/or decreasing the fraction of the population having an undesired size and/or sequence.

In the context of the present disclosure, “buffer” and “buffering agent” refer to a chemical entity or composition that itself resists and, when present in a solution, allows such solution to resist changes in pH when such solution is contacted with a chemical entity or composition having a higher or lower pH (e.g., an acid or alkali). Examples of suitable non-naturally occurring buffering agents that may be used in disclosed compositions, kits, and methods include HEPES, MES, MOPS, TAPS, tricine, and Tris. Additional examples of suitable buffering agents that may be used in disclosed compositions, kits, and methods include ACES, ADA, BES, Bicine, CAPS, carbonic acid/bicarbonic acid, CHES, citric acid, DIPSO, EPPS, histidine, MOPSO, phosphoric acid, PIPES, POPSO, TAPS, TAPSO, and triethanolamine.

In the context of the present disclosure, “cell-free” refers to a composition that contains no detectable viable cells. A cell-free composition, for example, may be free of living cells and still comprise one or more cellular components (e.g., products of cell lysis) and/or non-living cells (e.g., formalin fixed tissue specimens).

In the context of the present disclosure, “container” refers to a human-made container. A container may comprise one or more walls (e.g., defining an interior volume) and optionally one or more openings. Containers comprising one or more openings may further comprise one or more closures (e.g., a removable closures) for some or all such openings. A closure optionally may comprise an aperture or a septum, for example, to provide fluid communication with a volume of the container and an inserted tube or syringe. Examples of containers include boxes, cartons, bottles, tubes (e.g., test tubes, microcentrifuge tubes), plates (e.g., 96-well, 384-well plates), vials, pipette tips, and ampules. Containers and/or closures may comprise any desired material including paper, plastics, glass, silicone, composites, metals, alloys, or combinations thereof. Containers and/or closures may comprise materials that are compostable, recyclable, and/or sustainable.

In the context of the present disclosure, with respect to nucleotide bases in a double-stranded molecule, “correct” refers to pairs of bases on opposite strands that form Watson-Crick base pairs. Examples of correct pairings include the pairing of A and T of:
5′-NNANNN′3′
3′-NNTNNN′5′,
   in which the A and T form two hydrogen bonds, and the pairing of G and C of:
5′-NNGNNN′3′
3′-NNCNNN′5′,
   in which the G and C form three hydrogen bonds.

In the context of the present disclosure, “DNA fragment” refers to double-stranded or single-strand DNA, such as an oligonucleotide, that may be used in gene assembly. DNA fragments may be provided in any way desired. For example, DNA fragments may arise from annealing synthetically produced single strands or from the action of one or more restriction enzymes on synthetic or natural polynucleotides. DNA fragments may include both DNA substrates and error-free DNA fragments.

In the context of the present disclosure, “DNA substrate” refers to a double-stranded DNA comprising at least one type of error (e.g., at least one of a mismatch, an insertion, and a deletion). A DNA substrate may have, for example, at least on mismatch and at least one indel. A DNA substrate may arise from any desired source or synthesis method. For example, a DNA substrate may comprise a first single-stranded DNA annealed to a second single-stranded DNA, wherein each strand independently may be produced by an in vitro synthesis method and/or may arise from a natural source (with or without fragmentation, tailing, adapter ligation, editing, or other processing). DNA substrates may include, for example, dsDNA/PCR amplicons using templates from Cas9, TALEN, ZFN edited genome or chemically synthesized oligonucleotides. A DNA substrate may be linear or circular.

In the context of the present disclosure, “double-stranded” refers to a polynucleotide structure in which the bases of a first polynucleotide strand form Watson-Crick pairs with the bases of a distal region of the first polynucleotide strand (e.g., looped back on itself) or the bases of a second polynucleotide, in either case, positioned anti-parallel to the first polynucleotide strand. A double-stranded polynucleotide may comprise one or more mismatches and/or one or more indels. For example, a polynucleotide in which at least 50%, at least 60%, at least 70%, at least 80%, at least 90%, at least 95%, or at least 98% of the bases are Watson Crick paired may be referred to as double-stranded with the remaining bases unmatched (e.g., mismatch, indel, and/or overhanging bases). Double stranded DNA may be referred to as “dsDNA.”

In the context of the present disclosure, “endonuclease” refers to a nuclease that cleaves at least one internal phosphodiester bond of a duplex polynucleotide, wherein the bond cleaved is (a) at least one phosphodiester bond away from a 5′ terminal nucleotide and at least one phosphodiester bond away from a 3′ terminal nucleotide and (b) within 1-3 nucleotides of either an unpaired nucleotide (e.g., a component of an indel) or a nucleotide pair that does not form a Watson-Crick base pair (e.g., a mismatch). Examples of endonucleases include Endo VII and EndoMS. For clarity, in the context of this disclosure, endonucleases do not include Type I, II, IIG, IIP, IIS, III, or IV endonucleases except to the extent they meet this definition.

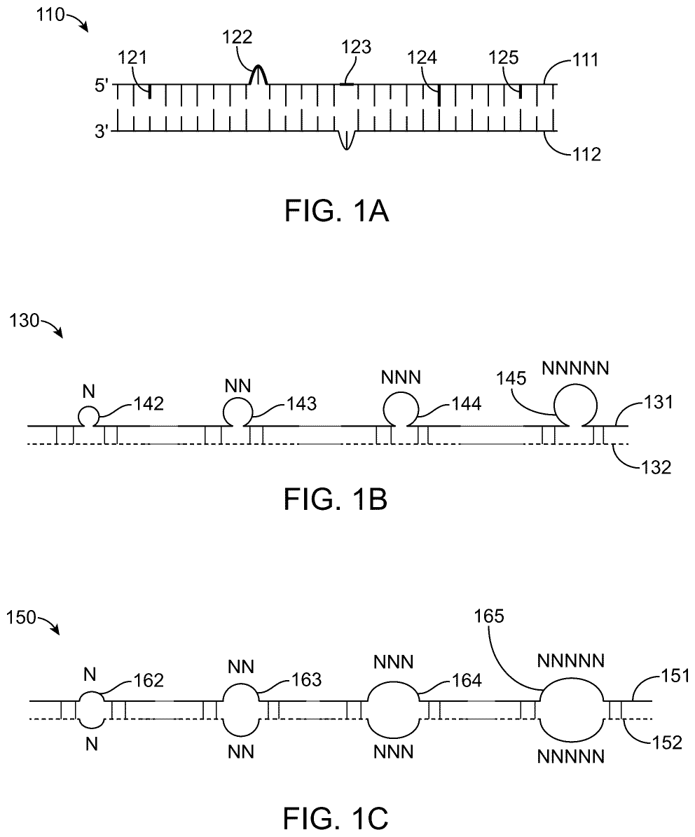

In the context of the present disclosure, “error” refers to any insertion, deletion, or mismatches and constitutes an error with respect to a reference sequence. In the context of a duplex polynucleotide, one strand may be deemed to comprise a sequence error with respect to the opposite strand or a reference sequence. A single-stranded polynucleotide may be said to comprise an error with respect to a reference sequence. Example errors are shown in FIG. 1A, FIG. 1B, and FIG. 1C.

In the context of the present disclosure, “T4EndoVII” refers to T4 endonuclease VII, an enzyme that recognizes and cleaves mismatches in heteroduplexes and looped or branched DNAs. The wild type enzyme has a mass of about 18 kDa and is encoded by gene 49 of T4 bacteriophage. T4EndoVII is involved in DNA-packaging, genetic recombination, and mismatch repair in vivo. In vitro T4EndoVII cleaves single-base mismatches, heteroduplex loops, and branched DNAs, such as four-way Holliday junctions and three-way Y structures. Examples of T4EndoVII cleavage are illustrated in FIG. 2A. Examples enzymes include the T4EndoVII endonucleases disclosed in Kemper B et al., “Studies on the function of gene 49 controlled endonuclease of phage T4 (endonuclease VII)” Prog Clin Biol Res. 1981; 64:151-66 and Mizuuchi K et al., “T4 endonuclease VII cleaves holliday structures” Cell. 1982 June; 29 (2): 357-65. For clarity, T4EndoVII does not include type IIS endonucleases. One unit of T4EndoVII, in some embodiments, is the amount of enzyme used to convert 0.5 μg of supercoiled pUC(AT) to linear DNA in 50 μL of 1× NEBuffer r2.1 reacted for 30 minutes at 37° C. A T4EndoVII may have at least 85%, at least 90%, at least 95%, at least 98%, at least 99%, or 100% identity with SEQ ID NO: 31.

In the context of the present disclosure, “T4EndoVII/EndoMS composition” refers to a composition comprising a first endonuclease and a second endonuclease, wherein the first endonuclease is T4EndoVII and the second endonuclease is EndoMS. A T4Endo VII/EndoMS composition need not comprise any additional nucleases, but may. A T4EndoVII/EndoMS composition may also comprise any of the other materials disclosed herein.

In the context of the present disclosure, “EndoMS” refers to any mismatch endonuclease of the conserved family of DNA mismatch endonucleases that are Mg2+-dependent and readily cleave the third phosphodiester bond on the 5′ side of T:T, G:G, and T:G mismatches leaving 5 nucleotide overhangs in both strands and also able to cleave DNA strands having T:I, G:I, and G:U mismatches. Examples of EndoMS cleavage are illustrated in FIG. 2B. Examples enzymes include the EndoMS endonucleases disclosed in U.S.

U.S. Pat. No. 11,371,088. For clarity, EndoMS does not include type IIS endonucleases. One unit of EndoMS, in some embodiments, is the amount of enzyme used to convert 0.5 μg of supercoiled pUC(AT) to linear DNA in 50 μL of 1× NEBuffer r2.1 reacted for 30 minutes at 37° C. An EndoMS may have at least 85%, at least 90%, at least 95%, at least 98%, at least 99%, or 100% identity with SEQ ID NO:32.

In the context of the present disclosure, an “error-free DNA fragment” is a ssDNA fragment having the exact length and sequence of a reference sequence (or optionally, the exact length of and complementarity to a reference sequence) or a dsDNA having a first strand and a second strand, the first and second strands having the exact length of and complementarity to each other.

In the context of the present disclosure, “gene assembly” refers to joining nucleic acids. Gene assembly may include joining DNA fragments (e.g., oligonucleotides) into polynucleotides. Gene assembly methods may include providing one or more oligonucleotides (e.g., through chemical synthesis and/or IVT) and assembling the oligonucleotides into polynucleotides.

In the context of the present disclosure, “homogeneous” refers to the property of sequence identity among members of a population of DNA molecules. For example, a first population of 100 DNA molecules all having the same size and sequence may be described as homogeneous and a second population of DNA molecules consisting of 90 molecules of the same size and sequence and 10 molecules that each differ from the 90 in size and/or sequence may be described as having 90 mole % homogeneity.

In the context of the present disclosure, “inconsistency” between two polynucleotides refers to any deviation (e.g., in size and/or sequence) from either perfect identity or perfect complementarity. For example, there is no inconsistency between 5′AATTCCGG3′ and 5′AATTCCGG3′ (which are identical) and there is no inconsistency between 5′AATTCCGG3′ and 5′CCGGAATT3′ (which are complementary), whereas 5′A^{m6}ATTCCGG3′ and 5′AATTCCGG3′ have an inconsistency at the second position and 5′AATTCCGG3′ and 5′CCGGAAATT3′ are inconsistent in both length and sequence. For clarity, inconsistencies include errors.

In the context of the present disclosure, “indel” refers to a region of a double-stranded DNA in which one or more contiguous nucleotides (e.g., 1-5, 1-10, 1-20, or 2-10) of one strand are missing relative to the opposing strand. For example, where a top strand of a duplex has n nucleotides and the bottom strand has n complementary nucleotides and one additional nucleotide along its length for a total of n+1 nucleotides, the top strand may be said to have a 1-nucleotide deletion relative to the bottom strand or the bottom strand may be said to have a 1-nucleotide insertion relative to the top strand. For clarity, the presence or omission of bases in one strand relative to the other may or may not be a consequence of an insertion or deletion event. For example, one or both strands may be synthetic or otherwise produced in vitro without the occurrence of any insertion or deletion event. The structures of indels include and may be described as or likened to a branch or loop as illustrated in FIG. 1A, FIG. 1B, and FIG. 1C.

In the context of the present disclosure, “mismatch” refers to nucleotide bases positioned opposite each other on opposing strands of a double-stranded DNA wherein the opposing nucleotide bases do not form a Watson-Crick base pair. Example mismatches include, without limitation, A:A, A:C, A:G, C:C, C:T, G:G, G:T, and T:T. Mismatches may exist between two canonical bases, a canonical base and a modified base, and two modified bases. Mismatches may be associated with structural and dynamic distortions of double-stranded DNA including, for example, dimensions of the grooves and frequency of breathing and/or associated with a glycosidic bond orientation (e.g., syn or anti). Unless context otherwise provides, a mismatch may include 1, 2, 3, 4, 5, 6, 7, 8, 9, or 10 contiguous bases in a strand that are not Watson-Crick base paired to their opposing bases in the opposite strand of a double-stranded DNA. For example, a DNA substrate may comprise a mismatch, wherein the mismatch comprises one base on a first strand of the DNA substrate and one opposing base on the strand opposite the first strand, wherein the two bases do not form a Watson-Crick base pair with each other but the bases immediately adjacent on both the 5′ and 3′ sides are paired (e.g., 5° . . . AAA . . . 3′/3′ . . . TGT . . . 5′). A DNA substrate may comprise a mismatch, wherein the mismatch comprises two bases on a first strand of the DNA substrate and two opposing bases on the strand opposite the first strand, wherein the four bases do not form a Watson-Crick base pair with each other but the bases immediately adjacent on both the 5′ and 3′ sides are paired (e.g., 5° . . . AAAA . . . 3′/3″ . . . TGGT . . . 5′). A mismatch may occur at any position along the length of a DNA (e.g., at the 5′ end, at the 3′ end, or at any base(s) between the 5′ end and the 3′ end). The structure of a mismatch may be described as or likened to a loop as illustrated in FIG. 1A, FIG. 1B, and FIG. 1C.

In the context of the present disclosure, “modified nucleoside” refers to nucleosides having a modification on the sugars (e.g., 2′-fluororibose, ribose, 2′-deoxyribose, arabinose, and hexose); and/or in the nucleotide base (e.g., as described in U.S. Pat. No. 8,383,340: WO 2013/151666; U.S. Pat. No. 9,428,535 B2; US 2016/0032316). Modified nucleosides include adenosine analogs, uridine analogs, guanosine analogs, and cytidine analogs.

In the context of the present disclosure, “modified nucleotide” refers to nucleotides having a modification on the sugars (e.g., 2′-fluororibose, ribose, 2′-deoxyribose, arabinose, and hexose); and/or in the phosphate groups (e.g., phosphorothioates and 5′-N-phosphoramidite linkages); and/or in the nucleotide base (e.g., as described in U.S. Pat. No. 8,383,340; WO 2013/151666; U.S. Pat. No. 9,428,535 B2; US 2016/0032316).

In the context of the present disclosure, “non-naturally occurring” refers to a molecule (e.g., a polynucleotide, polypeptide, carbohydrate, or lipid) or composition that does not exist in nature. Such a molecule or composition may differ from naturally occurring molecules or compositions in one or more respects. For example, a polymer (e.g., a polynucleotide, polypeptide, or carbohydrate) may differ in the kind and arrangement of the component parts (e.g., nucleotide sequence, amino acid sequence, or sugar molecules). A polymer may differ from a naturally occurring polymer with respect to the molecule(s) to which it is linked. For example, a “non-naturally occurring” polypeptide (e.g., protein) may differ from naturally occurring polypeptides in its secondary, tertiary, or quaternary structure, by having (or lacking) a chemical bond (e.g., a covalent bond including a peptide bond, a phosphate bond, a disulfide bond, an ester bond, and ether bond, and others) to a lipid, a carbohydrate, a second polypeptide (e.g., a fusion protein), or any other molecule. Similarly, a “non-naturally occurring” polynucleotide or nucleic acid may comprise (or lack) one or more other modifications (e.g., an added label or other moiety) to the 5′-end, the 3′ end, and/or between the 5′- and 3′-ends (e.g., methylation) of the nucleic acid. A “non-naturally occurring” molecule or composition may differ from naturally occurring compositions in one or more of the following respects: (a) having components that are not combined in nature, (b) having components in ratios and/or concentrations not found in nature, (c) lacking one or more components otherwise found in naturally occurring molecules or compositions (e.g., a cell-free composition, a chromosome-free composition, a histone-free composition, a polymerase-free composition, a cell membrane-free composition), (d) having a form not found in nature (e.g., dried, freeze dried, lyophilized, crystalline, aqueous, immobilized), and (e) having one or more additional components beyond those found in nature (e.g., a buffering agent, a detergent, a dye, a solvent or a preservative).

In the context of the present disclosure, “oligonucleotide” refers to deoxyribonucleotides that are no more than 5000 nucleotides long or no more than 750) nucleotides long or no more than 500 nucleotides long or no more than 250 nucleotides long or no more than 200 nucleotides long or no more than 150 nucleotides long or no more than 100 nucleotides long. For example, oligonucleotides may be 4-80 nucleotides long, 4-60 nucleotides long, or 4-40 nucleotides long.

In the context of the present disclosure, a “proofreading polymerase” is a DNA polymerase having (a) the capacity to excise an incorrectly paired nucleotide at a strand terminus and adding the correct nucleotide in its place, and/or (b) the capacity to excise an unpaired nucleotide (e.g., an overhang or insertion) at a strand terminus. Proofreading activity may include 3′-+5′ exonuclease activity. Examples include Vent® DNA Polymerase, Deep Vent® DNA Polymerase, 9°Nm™ DNA Polymerase, Phusion® High-Fidelity DNA Polymerase, Q5 ® High-Fidelity DNA Polymerase, phi29 DNA polymerase, E. coli DNA polymerase I. T4 DNA polymerase, and DNA polymerase I, large (Klenow) fragment (New England Biolabs, Inc., Ipswich, MA, #0254, M0203, M0209, M0210, M0257, M0258, M0259, M0260, M0530, M0535, M0491, M0493, and M0269).

All publications, patents, and patent applications mentioned in this specification are herein incorporated by reference to the same extent as if each individual publication, patent, or patent application was specifically and individually indicated to be incorporated by reference.

Disclosed reagents were obtained from the indicated supplier or, if no supplier is indicated, from New England Biolabs, Inc., Ipswich, MA.

### Compositions

The present disclosure, in some embodiments, relates to compositions for cleaving polynucleotides comprising one or more errors. For examples, compositions of the disclosure may be adapted for cleaving polynucleotides comprising one or more errors with limited or no sequence context bias. Compositions may have any desired form including, for example, a cell-free, dried, freeze dried, lyophilized, crystalline, aqueous, liquid, or immobilized form. A composition may comprise, in some embodiments, non-naturally occurring combinations of endonucleases, for example, two or more endonucleases, wherein (a) at least one of the endonucleases is a non-naturally occurring endonuclease, and/or (b) at least two of the included endonucleases are from different sources. Naturally occurring sources of endonucleases include phage, bacteria, yeast, and plants and non-naturally occurring sources include cell-free expression systems, engineered phage, engineered bacteria, engineered yeast, and engineered plants. A first endonuclease may be a phage (e.g., T4) endonuclease and a second endonuclease may be an archaeal endonuclease (e.g., Pyrococcus) or an engineered variant thereof. In some embodiments, a composition may comprise T4 endonuclease VII and mismatch endonuclease I.

In some embodiments, compositions may include endonucleases at any desired concentration. For optimal cleavage activity of a substrate, it may be desirable for a composition to comprise one or more of the at least two or more endonucleases at a concentration higher (e.g., ≥10% higher, ≥25% higher, ≥50% higher, ≥70% higher, ≥ 100% higher, ≥200% higher, ≥500% higher, ≥1000% higher, or ≥2000% higher) than the concentration found in any natural source. A composition may comprise, for example, ≥0.1 U/mL T4EndoVII, ≥0.1 U/mL EndoMS, or both ≥0.1 U/mL T4EndoVII and ≥0.1 U/mL EndoMS. In some embodiments, compositions may include endonucleases at any desired ratio to one another. For example, a ratio (e.g., a unit ratio or a molar ratio) of T4EndoVII to EndMS may be from 1:50, 1:25, 1:10, 1:5, or 1:2 to 2:1, 5:1, 10:1, 25:1, or 50:1.

In some embodiments, compositions may include at least one additional enzyme (e.g., beyond endonucleases). For example, an endonuclease composition may further comprise a polymerase (e.g., Q5®), phi29), a ligase, a glycosylase (e.g., UDG), and/or a base editor. According to some embodiments, compositions may be free of one or more specified enzymes. For example, an endonuclease composition may be free of any or all polymerases (e.g., Q5®, phi29), any or all ligases, any or all glycosylases (e.g., UDG), and/or any or all base editors.

Compositions may include, according to some embodiments, one or more of the components included in a kit (e.g., as described below). In some embodiments, compositions may include one or more non-enzymatic components. For example, compositions may include a buffer or a buffering agent. In some embodiments, compositions may include one or more nucleotide triphosphates (NTPs). In some embodiments, NTPs may be dNTPs, rNTPs, or both. dNTPs may include one, two, three of all four of dATP, dTTP, dGTP and dCTP. rNTPs may include one, two, three of all four of rATP, rUTP, rGTP and rCTP. In some embodiments, NTPs may include one or more modified nucleosides and/or one or more modified nucleotides (e.g., as a dNTP or rNTP). Compositions may be free of one or more non-enzymatic components. For example, compositions may be free of any or all animal products (e.g., bovine serum albumin), free of any or all detergents (e.g., Triton X-100, polysorbate 20), free of glycerol, free of lipids, and/or free of carbohydrates. Compositions lacking one or more of these components may have improved properties including, for example, improved substrate, product, and/or enzyme stability, improved catalytic activity, and/or improved efficiency.

In some embodiments, compositions may include one or more polynucleotides. For example, compositions may include one or more DNA fragments, which may be substrates and/or products of authentication. DNA fragments may include homoduplex dsDNA, heteroduplex dsDNA, or combinations of homoduplex dsDNA and heteroduplex dsDNA. Authentication substrate DNA may include heteroduplex dsDNA having at least one error, each of which may be 1-10 bases in length. Compositions may include one or more polynucleotide primers. Primers may include a sequence complementary to a target (e.g., a binding target for amplification), a barcode, a restriction site, a linker and/or any other desired sequence.

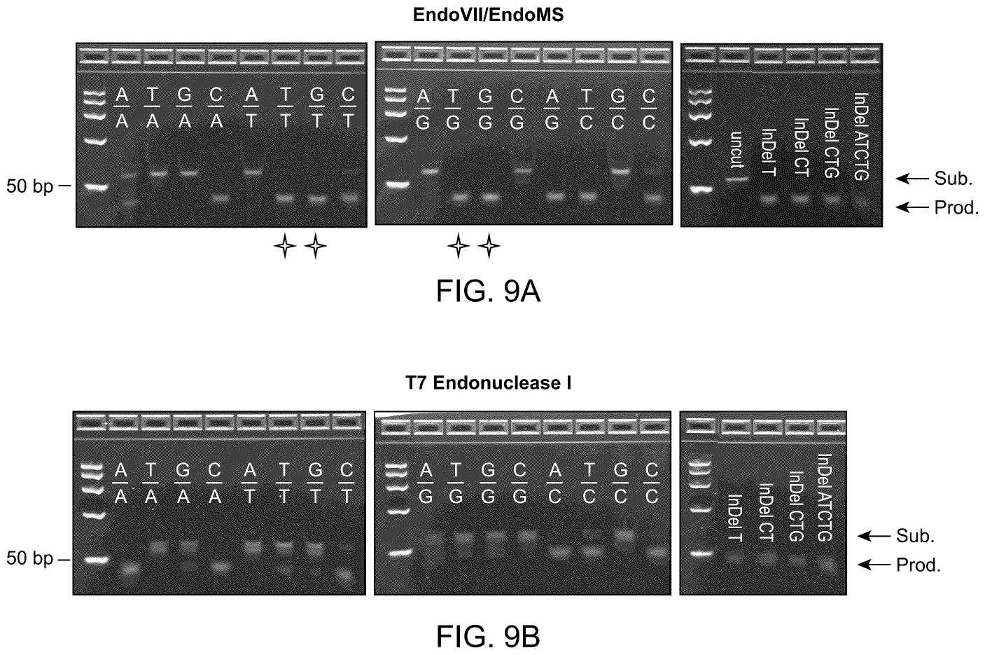

Cleavage by the T4EndoVII and EndoMS may be specific to heteroduplex DNA, with cleavage of homoduplex DNA (the form in which error-free DNA fragments may be present) absent (e.g., as shown FIG. 9A).

### Kits

The present disclosure, in some embodiments, relates to kits for authenticating a DNA. A kit may be a non-natural collection of components configured, for example, for convenient storage, shipping, delivery, and/or use. One or more components of a kit may be included in one container for a single step reaction, or one or more components may be contained in one container, but separated from other components for sequential use or parallel use. The contents of a kit may be formulated for use in a desired method or process.

For example, a kit may include one or more components to make one or more disclosed composition with each component in a separate volume or partially combined in two or more volumes (e.g., two of three components combined and one separate, two of four combined, one separate, three of four combined and one separate, and so on). A kit may include two or more endonucleases (e.g., T4EndoVII and/or EndoMS), each in a separate volume or combined in a single volume (e.g., a mastermix). A kit with endonucleases may further comprise one or more additional enzymes and/or one or more non-enzymatic components, for example, as described above in the context of compositions. For example, a kit may further include a buffering agent, other enzymes (e.g., polymerases, ligases, base editors), or combinations thereof. Enzymes may be included in a storage buffer (e.g., comprising glycerol and a buffering agent or a glycerol-free buffering agent). A kit may include (e.g., separately or combined with a buffing agent) additives (e.g. glycerol), salt (e.g. KCl, NaCl, MgCl₂), reducing agent, EDTA, detergents, modified amino acids (e.g., betaine), NTPs, and combinations thereof. For example, a kit may include a reaction buffer (optionally in concentrated form) comprising one or more additives (e.g. glycerol), salts (e.g. KCl, NaCl, MgCl₂), reducing agents, EDTA, detergents, or combinations thereof. A kit comprising dNTPs may include one, two, three of all four of dATP, dTTP, dGTP and dCTP. In some embodiments, a kit may include one or more modified nucleosides and/or one or more modified nucleotides (e.g., as a dNTP or rNTP). A kit may further comprise one or more modified nucleotides.

One or more kit components may be in a separate volume or container (e.g., one component in a single tube or in a single volume divided into n aliquots in n containers, where n=2−100, 1−1000, or more than 1000). One or more kit components may be combined in a single volume (e.g., two components combined in a single volume in a single container or combined in a single volume aliquoted into separate containers). For example, one or more components of a kit may be included in one container for a single step reaction (e.g., a master mix including all enzymes, NTPs, ions, and buffers in a single container in anticipation of a user adding a substrate). One or more components may be contained in one container but separated from other components for sequential use or parallel use (e.g., a first enzyme with its reaction buffer included. The contents of a kit may be formulated for use in a desired method or process.

A kit is provided that contains: (i) a T4EndoVII; (ii) an EndoMS; and (iii) a buffering agent. One or both enzymes may have a lyophilized form or may be included in a buffer (e.g., a storage buffer or a reaction buffer in concentrated form). A kit may contain either or both enzymes in a master mix suitable for cleaving a DNA. Either of or both T4EndoVII and EndoMS may be a purified enzyme so as to contain substantially no DNA or RNA and no other nucleases and may also be present in a cell-free composition. A reaction buffer in (iii) and/or storage buffers containing the enzymes in (i) and/or (ii) may include non-ionic, ionic e.g. anionic or zwitterionic surfactants and crowding agents. A kit may include T4EndoVII, EndoMS, and the reaction buffer in a single tube or in different tubes.

A subject kit may further include instructions for using the components of the kit to practice a desired method. The instructions may be recorded on a suitable recording medium. For example, instructions may be printed on a substrate, such as paper or plastic, etc. As such, the instructions may be present in the kits as a package insert, in the labeling of the container of the kit or components thereof (i.e., associated with the packaging or sub-packaging) etc. Instructions may be present as an electronic storage data file residing on a suitable computer readable storage medium (e.g. a CD-ROM, a flash drive). Instructions may be provided remotely using, for example, cloud or internet resources with a link or other access instructions provided in or with a kit.

### Methods

The present disclosure relates, in some embodiments, to authenticating polynucleotides. For example, methods are provided for identifying and/or correcting errors in one or more polynucleotides. Methods may include contacting one or more polynucleotides comprising one or more errors (per population or per molecule) with two or more endonucleases optionally from different sources and optionally with different specificities (e.g., T4EndoVII and EndoMS) to produce cleavage products defined, at least in part, by site(s) of heterogeneity (e.g., the errors). For example, contacting (a) dsDNA having a first and second strand, wherein the first and second strand are less than perfectly complementary, for example, where together they comprise one or more errors, with (b) two or more endonucleases may produce a nicked dsDNA cleavage product, wherein the first strand is separated into a 5′ fragment and a 3′ fragment the error defining either the 3′ end of the 5′ fragment or the 5′ end of the 3′ fragment. Identifying polynucleotides having one or more errors may further comprise analyzing (e.g., monitoring) cleavage products for the appearance of nicked or cleaved DNA, optionally over time or at any time, through any available technique including, for example, size fractionation, fluorescent and/or affinity labeling. Correcting errors may further comprise contacting cleavage products with one or more proofreading polymerases.

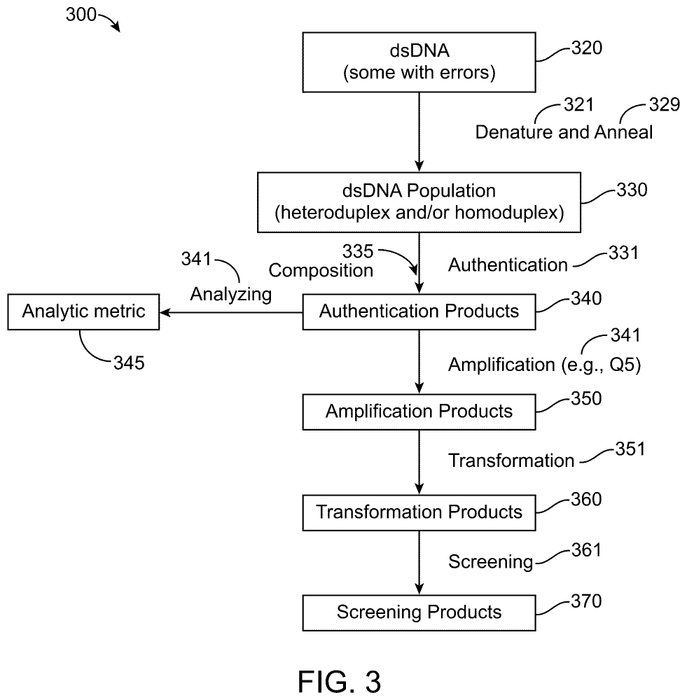

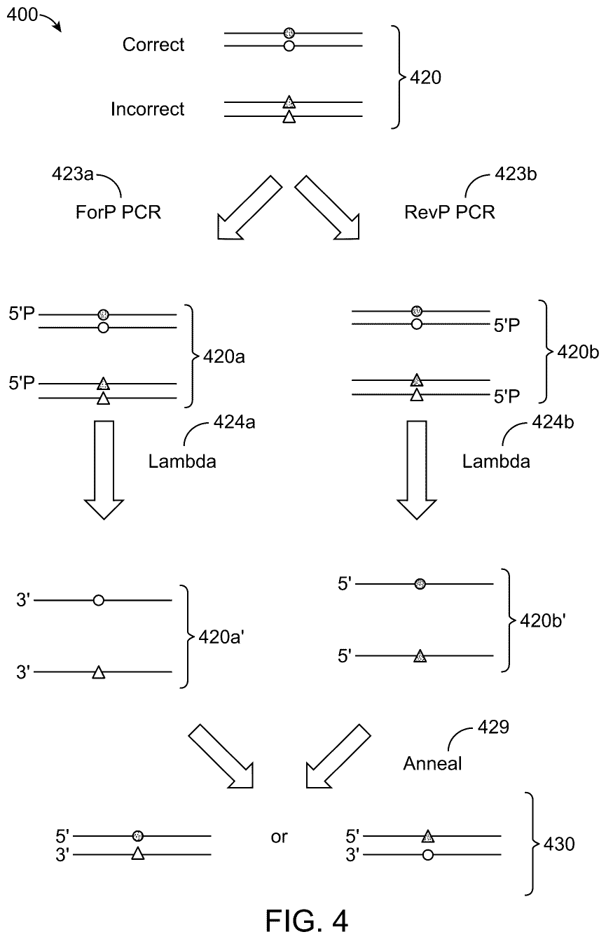

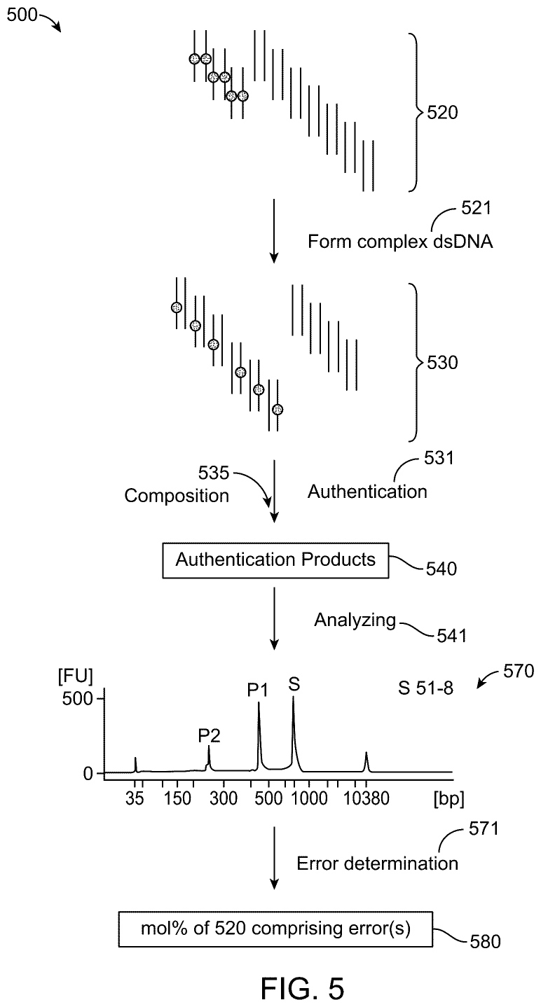

The present disclosure provides methods for authenticating DNA, for example, method 300 illustrated in FIG. 3. Method 300 may comprise forming heteroduplexes, wherein strands of DNA in a population of double-stranded homoduplex DNA each exchange their initial hybridization partner with a new partner from the population. The sequence of the new partner strand may be the same as the original partner (forming a new homoduplex) or different from the original partner strand (forming a heteroduplex). Heteroduplex DNA may be formed when the original population is heterogenous with some (e.g., ≥85%, ≥90%. ≥95%, ≥97.5%) of the species having a desired sequence and some (e.g., ≤15%, ≤10%, ≤5%, ≤2.5%) of the species having one or more errors relative to the desired sequence. Heteroduplex formation may comprise forming a population of complex dsDNA molecules 330, for example, by denaturing 322 dsDNA 320 to form separated strands and annealing 329 separated strands to form population 330. Population 330 may comprise homoduplex DNA molecules, heteroduplex DNA molecules or both homoduplex DNA molecules and heteroduplex DNA molecules depending, at least in part, on the homogeneity of species in the original population 320. For example, if all DNA species in original population 320 are 100% identical in length and sequence, population 330 should consist of homoduplex DNA. If, on the other hand, population 320 comprises molecules having one or more differences in size and/or sequence, population 330 should consist of homoduplex DNA and heteroduplex DNA. It will be appreciated that homoduplexes may not be substrates for authentication. While this may be acceptable for homoduplexes of the desired sequence, it may be undesirable where original duplexes comprising an error relative to the desired sequence reform during annealing 329. In some embodiments, population 320 may be prepared to reduce or minimize formation of authentication resistant duplexes as described herein (e.g., method 400 illustrated in FIG. 4 and method 500 illustrated in FIG. 5). Method 300 may comprise authenticating 331 complex population of dsDNA molecules 330 optionally comprising contacting complex population 330 with composition 335 to produce authentication products 340, wherein authentication products 340 may comprise one or more mismatch cleavage products and/or one or more indel cleavage products. Composition 335 may be any of the compositions disclosed in this specification, for example, a composition comprising endonucleases (e.g., T4EndoVII and EndoMS). Composition 335 may be a component or a combination of components of any of the kits disclosed herein. During authentication 331. T4EndoVII and/or EndoMS of composition 335 may recognize one or more errors, if present, in population 330 and cleave a phosphodiester bond at the site of such errors to form a nicks or double-stranded breaks (e.g., with or without overhangs). In some embodiments, cleavage products may include fragments corresponding in size (e.g., +5 nucleotides) to the number of nucleotides from a 5′ end of the DNA substrate to an error, from one error to an adjacent error, from an error to a 3′ end of the DNA substrate, or combinations thereof.

Method 300 may comprise amplifying 341 authentication products 340 (in total) or one or more species included therein. Amplifying 341 may include, for example, contacting authentication products 340 or one or more species included therein with a polymerase, for example, with a high-fidelity polymerase (e.g., Q5® High-Fidelity DNA Polymerase, which has 3′→5′ exonuclease activity that excises mismatches and/or cuts back 3′ overhangs), to produce amplification products 350. Contacting may further include contacting authentication products 340 or one or more species included therein with a polymerase (and, optionally, dNTPs and/or primers. Method 300 may comprise transforming 351 one or more cells with amplification products 350 to produce one or more transformation products 360. Method 300 may comprise screening 361 one or more transformation products 360 to produce one or more screening products 370.

In some embodiments, it may be desirable to detect the presence of one or more errors in a population of DNA molecules with or without further analysis of the error and/or with or without correction of the error. Method 300 may be used for each of these applications. As shown, method 300 may comprise analyzing 341 authentication products 340 to produce one or more analytic products 345 including, for example, information regarding the presence and/or quantity of one or more mismatch cleavage products and/or the presence and/or quantity of one or more indel cleavage products. Analytic products 345 may include data, results, or other information on one or more properties of authentication products 340 including, for example, the presence of one or more (e.g., endonuclease) cleavage products, the actual and/or relative size of one or more DNA molecules in the population of products, the nucleotide sequence of one or more DNA molecules in the population, and/or the actual and/or relative concentration of one or more homoduplex molecules in the population of products.

It will be recognized that each of amplifying 341 and analyzing 345 may be performed on a portion (e.g., an aliquot) of authentication products 340, leaving a remainder of authentication products 340 for other uses including, for example, analyzing 345 and amplification 341, respectively. In some embodiments, method 300 may comprise denaturing 328, reannealing 329, and authentication 331 with or without additional steps shown. Method 300 may comprise, according to some embodiments, analyzing 341 with or without additional steps shown. In some embodiments, method 300 may comprise amplifying 341, transforming 351, and screening 361 with or without additional steps shown.

Method 300 may be applied to any population of DNA molecules including, for example, genomic DNA (e.g., fragmented genomic DNA), plasmid DNA, cDNA, chemically synthesized DNA (e.g., oligonucleotides), assembled DNA (e.g., products of Golden Gate Assembly or NEBuilder® HiFi DNA Assembly (NEB. Inc., Ipswich. MA)), and/or PCR amplicons. In some embodiments. PCR amplicons may be from a Cas9, TALEN, ZFN, or other base-edited genome (e.g., edited by using an adenine base editor or a cytidine base editor). In some embodiments, a population of DNA molecules for method 300 may comprise or consist of DNA purified from a phage, other virus, bacteria, yeast, other fungus, protozoa, algae, other plant, mammal, or other animal. Method 300 may begin with an existing population of DNA molecules or may comprise (e.g., in one or more steps) forming the population of DNA molecules. For example, method 300 may include forming a population of DNA molecules by chemical synthesis, amplification (e.g., PCR), editing (base editing or prime editing) one or more DNA molecules, or any combinations thereof.

Methods of the present disclosure, including method 300, may yield populations of DNA molecules with greater homogeneity and/or fewer errors than the starting population, according to some embodiments. For example, DNA populations, including population 350, may contain a higher proportion of error-free DNA molecules and/or a lower number of errors per nucleotide than would be achieved by methods of contacting the original population a single endonuclease (e.g., with only T4EndoVII or only EndoMS). DNA populations, including population 350, may be substantially free of molecules comprising errors, for example, with ≤1 molecule of 10 comprising an error, ≤8 molecules of 100 comprising an error, ≤5 molecules of 100 comprising an error, ≤2 molecules of 100 comprising an error, ≤1 molecule of 100 comprising an error, ≤8 molecules of 1,000 comprising an error, ≤5 molecules of 1,000 comprising an error, ≤2 molecules of 1,000 comprising an error, ≤1 molecule of 1,000 comprising an error, ≤8 molecules of 10⁴ comprising an error, ≤5 molecules of 10⁴ comprising an error, ≤2 molecules of 10⁴ comprising an error, ≤1 molecule of 10⁴ comprising an error, ≤8 molecules of 10⁵ comprising an error, ≤5 molecules of 10⁵ comprising an error, ≤2 molecules of 10⁵ comprising an error, ≤1 molecule of 10⁵ comprising an error, ≤8 molecules of 10⁶ comprising an error, ≤5 molecules of 10⁶ comprising an error, ≤2 molecules of 10⁶ comprising an error, or ≤1 molecule of 10⁶ comprising an error. Populations of DNA molecules, including population 350, may be substantially free of errors, for example, ≤1,000 errors per 10⁶ nucleotides, ≤500 errors per 10⁶ nucleotides, ≤250 errors per 10⁶ nucleotides, ≤100 errors per 10⁶ nucleotides, ≤50 errors per 10⁶ nucleotides, ≤10 errors per 10⁶ nucleotides, ≤5 errors per 10⁶ nucleotides, ≤2 errors per 10⁶ nucleotides, ≤1 error per 10⁶ nucleotides, ≤100 errors per 109 nucleotides, ≤50 errors per 109 nucleotides, or ≤10 errors per 109 nucleotides.

In some embodiments, amplification may include contacting the authentication products with a polymerase, specifically a DNA polymerase, and NTPs. Amplification may include PCR amplification of authentication products in the presence of DNA polymerase, NTPs, and primers. Amplification products, in some embodiments, may comprise or consist of dsDNA. dsDNA amplification products may form a DNA population, which may have any proportions of error-free DNA fragments as described above in connection with the authentication products (e.g. at least 90% error-free DNA fragments).

In some embodiments, a method may comprise a transformation step (e.g., transforming 351) that comprises ligating error-corrected, amplified DNA (e.g., amplification products 350) into a vector that allows expression in the type of cell to be transformed. A vector may further include at least one selection gene used in subsequent screening (e.g., screening 361). In some embodiments, error-corrected, amplified DNA fragments may be assembled to form an artificial gene as the amplification product. In some embodiments, the cell may be a bacterial cell and the transformation products may include a population of bacteria, some of which comprise DNA fragments, and some of which do not (e.g., bacteria that are not successfully transformed). In some embodiments, the cell may be a yeast and the transformation products may include a population of yeast, some of which comprise DNA fragments, and some of which do not (e.g., yeast that are not successfully transformed).

Screening transformation products (e.g., screening 361) may include growing transformation products (e.g., transformation products 360) in conditions that allow the isolation of a colony of cells derived from a single transformation product cell, such as plating on agar containing an appropriate selection medium. Colonies are picked and analyzed for presence of correct clones. In some embodiments, colonies may be analyzed directly by colony PCR with appropriate primers followed by size fractionation (e.g., agarose gel electrophoresis), Sanger or next generation sequencing of the amplicon, or both. In some embodiments, colonies are analyzed indirectly by analysis of miniprep plasmid DNA after overnight culture using restriction enzyme digestion, Sanger or next-generation sequencing, or both. The resulting screening products may include colonies or vectors that have been successfully transformed with error-free DNA fragments.

Transformation methods that do not include authentication as disclosed herein would generally be expected to result in transformation products having a relatively high proportion of errors in the introduced sequences and/or a relatively low number of successful (error-free) products. Method 300 results in a comparatively higher proportion of cells that have been transformed with error-free DNA fragments. For example, in method 300, the proportion of cells in the screening product that are transformed with error-free DNA fragments is similar to the proportions of error-free DNA fragments described above as in connection with authentication products (e.g., at least 90% or screening product cells contain only error-free DNA fragments).

In some embodiments, it may be necessary or desirable to have screening products comprising introduced sequences that are substantially error-free or error-free. In such cases, methods of the disclosure, including method 300, increase the proportion of usable products. Process efficiency gains are realized by requiring less colony-picking and sequencing of screening product samples.

The present disclosure further provides methods for reducing or eliminating authentication-resistant duplexes in authentication substrates, for example, method 400 illustrated in FIG. 4. As shown, in the population of dsDNA 420, some members have a correct or desired nucleotide pair at a given position (shown by circles) while other members have an incorrect or undesired nucleotide pair at the given position (shown by triangles). It will be appreciated that simply melting an reannealing dsDNA 420 may result in formation of heteroduplexes comprising a circle:triangle mismatch and/or reformation of circle:circle and triangle:triangle homoduplexes. The reformed circle:circle duplexes have the desired sequence and may, therefore, be of little concern. The reformed triangle:triangle duplexes, on the other hand, may be concerning since they may be resistant to subsequent authentication. Method 400 may comprise amplifying 423a a first aliquot of dsDNA 420 with a phosphorylated forward amplification primer and unphosphorylated reverse primer to produce amplification products 420a having 5′ phosphates on the top strands and amplifying 423b a second aliquot of dsDNA 420 with an unphosphorylated forward amplification primer and phosphorylated reverse primer to produce amplification products 420b having 5′ phosphates on the bottom strands. Method 400 may comprise contacting 424a a lambda exonuclease and amplification products 420a to produce digestion products 420a′ and independently may comprise contacting 424b a lambda exonuclease and amplification products 420b to produce digestion products 420b′. Method 400 may comprise annealing 429 digestion products 420a′ and 420b′ to form a dsDNA population of complex dsDNA molecules 430. All or substantially all the errors in strands of population 420 may be paired in heteroduplexes in population 430. For example, ≤0.00001%, ≤0.00005%, ≤0.0001%, ≤0.0005%, ≤0.001%, ≤0.005%, ≤0.01%, ≤0.05%, ≤0.1%, ≤0.5%, ≤1%, ≤5%, or ≤10% of the errors of population 420 may be present in homoduplexes in population 430. For example, ≥90%, ≥95%, ≥98%, ≥99%, ≥99.5%, ≥99.9%, ≥99.95%, ≥99.99%, ≥99.995%, ≥99.999%, ≥99.9995%, ≥99.9999%, ≥99.99995%, or ≥99.99999% of the errors of population 420 may be present in heteroduplexes (e.g., comprising at least one mismatch and/or indel) in population 430 and, therefore, susceptible to authentication. Annealing 429 may further comprise purifying digestion products 420a′ to produce purified ssDNA 420a″ (not shown), purifying digestion products 420b′ to produce purified ssDNA 420b″ (not shown) and contacting purified ssDNA 420a″ and 420b″ to produce the annealed dsDNA population of dsDNA molecules 430.

The present disclosure further provides methods for assessing the prevalence of molecules comprising one or more errors in a dsDNA population, for example, method 500 illustrated in FIG. 5. As shown, method 500 may comprise forming 521 a population of complex dsDNA molecules 530, for example, by denaturing dsDNA 520 to form separated strands and annealing separated strands to form population 530. Method 500 may comprise authenticating 531 complex population of dsDNA molecules 530, wherein authenticating 531 optionally comprises contacting complex population 530 with a composition 535 to produce authentication products 540, wherein authentication products 540 may comprise one or more mismatch cleavage products and/or one or more indel cleavage products. Composition 535 may be any of the compositions disclosed in this specification, for example, a composition comprising endonucleases (e.g., T4Endo VII and EndoMS). Composition 535 may be a component or a combination of components of any of the kits disclosed herein. During authentication 531, T4EndoVII and/or EndoMS of composition 535 may recognize one or more errors, if present, in population 530 and cleave a phosphodiester bond at the site of such errors to form a nick.

Method 500 may further comprise analyzing 541 at least a portion of authentication products 540, for example, by size fractionation (e.g., Bioanalyzer as shown or gel electrophoresis) to produce output 570 showing quantity (moles) and relative size of substrate (“S”), a first product fragment (“P1”), and a second product fragment (“P2”). Method 500 may further comprise processing 571 to produce results 580 from output 570. For example, processing may include determining the mole fraction of dsDNA 520 comprising errors. For example, the mole percentage of heteroduplex molecules in complex population 530 may be given by Formula I:

$$\% heteroduplex = [ P 1 / ( P 1 + S ) ] , \qquad ( I )$$

wherein P1 is the number (e.g. moles) of one size of cleaved DNA substrate fragment and S is the number (e.g. moles) of the uncleaved DNA fragments (i.e. the error-free DNA fragments). Mole percentage of dsDNA 520 comprising errors may be given by Formula II:

$$\% Error = 100 \times [ 1 - ( \sqrt{1 - \% Heteroduplex} ) ] \qquad ( II )$$

As elaborated in EXAMPLE 2, the calculated mole percent of dsDNA 520 comprising one or more errors is 30%, based on the example conditions assayed in the example.

TABLE 1 shows an example chart of molarity versus DNA fragment size generated by analysis of an agarose gel, with P1 and S indicated. P2 is the molarity of the other fragment resulting from the cleavage that generated the P1 fragments (e.g. P2 is the other part of the cleaved DNA).

TABLE 1

| | | Size | Conc | Molarity | |
|---|---|---|---|---|---|
| | | (bp) | (pg/μL) | (pmol/L) | Notes |
| 1 | [symbol] | 35 | 125.00 | 5,411.3 | Lower Marker |
| 2 | | 142 | 20.31 | 217.2 | |
| 3 | | 151 | 7.99 | 80.2 | |
| 4 | | 168 | 7.27 | 65.7 | |
| 5 | | 175 | 16.05 | 138.8 | |
| 6 | | 204 | 41.03 | 305.1 | |
| 7 | | 216 | 22.38 | 156.7 | |
| 8 | | 244 | 42.36 | 262.7 | |
| 9 | | 251 | 228.18 | 1,378.0 | P2 |
| 10 | | 290 | 8.50 | 44.4 | |
| 11 | | 306 | 15.55 | 77.1 | |
| 12 | | 339 | 9.26 | 41.4 | |
| 13 | | 350 | 9.10 | 39.4 | |
| 14 | | 389 | 25.84 | 100.7 | |
| 15 | | 414 | 19.85 | 72.6 | |
| 16 | | 447 | 509.56 | 1727.8 | P1 |
| 17 | | 489 | 40.62 | 125.8 | |
| 18 | | 523 | 14.46 | 41.8 | |
| 19 | | 582 | 34.71 | 90.4 | |
| 20 | | 683 | 647.42 | 1,436.4 | S |
| 21 | [symbol] | 10,380 | 75.00 | 10.9 | Upper Marker |
| 22 | | 12,010 | 0.00 | 0.0 | |

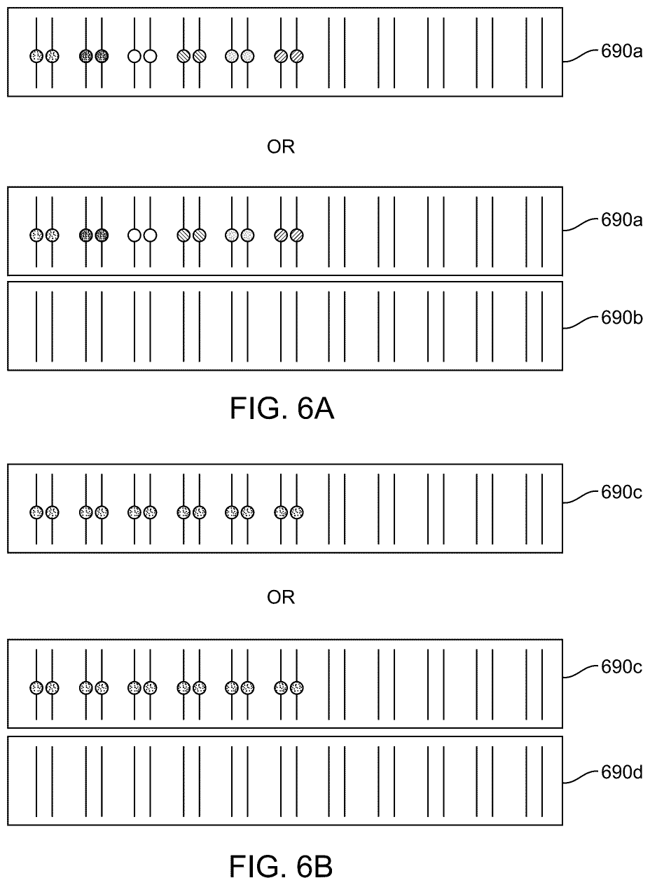

FIG. 6A and FIG. 6B illustrate example populations of dsDNA that may be assayed and/or authenticated in accordance with embodiments of the disclosure. FIG. 6A illustrates a first population of dsDNA 690a comprising members with one of six different errors (circles) (each constituting 10 mole % of the population) and members with the desired sequence (totaling 40 mole % of the population). FIG. 6A illustrates a second population of dsDNA 690b comprising a homogenous population of members having the desired sequence (100 mole %) combined with first population of dsDNA 690a. Analysis of population 690a alone by sequencing may be expected to correctly reveal that 60 mole % of population 690a comprises an error. Analysis of population 690a alone by method 500 may produce a calculation of 53.1 mole %. By comparison, if population 690a is first combined with population 690b in equimolar amounts, method 500 may produce a more accurate calculation of 57.9 mole %.

FIG. 6B illustrates a first population of dsDNA 690c comprising members with six copies of an error (circles) (totaling 60 mole % of the population) and members with the desired sequence (totaling 40 mole % of the population). FIG. 6B illustrates a second population of dsDNA 690d comprising a homogenous population of members having the desired sequence (100 mole %) combined with first population of dsDNA 690c. Analysis of population 690c alone by sequencing may be expected to correctly reveal that 60 mole % of population 690c comprises an error. Analysis of population 690c alone by method 500 may produce a calculation of 27.9 mole %. By comparison, if population 690c is first combined with population 690d in equimolar amounts, method 500 may produce a more accurate calculation of 47.7 mole %.

Examples of methods for authenticating dsDNA may include method 700 illustrated in FIG. 7. As shown, method 700 may comprise optionally assembling 711 oligonucleotide fragments 710. As shown, some mole fraction of oligonucleotide fragments 710a, 710b, 710c, and 710d present in the assembly reaction have an error (E). The unnumbered fragments are present in homogenous populations of the desired sequence. Fragments include forward primer 710z and reverse primer 710z′. Method 700 may comprise forming heteroduplexes as described in connection with method 300 and FIG. 3. For example, method 700 may comprise denaturing 721 dsDNA 720 to form separated strands and annealing 729 separated strands to form a dsDNA population of dsDNA molecules 730, which may comprise homoduplex DNA molecules, heteroduplex DNA molecules or both homoduplex DNA molecules and heteroduplex DNA molecules. Method 700 comprises authenticating 731 complex population of dsDNA molecules 730 optionally comprising contacting population 730 with a composition comprising endonucleases, for example, T4EndoVII and EndoMS, to produce authentication products 740, wherein authentication products 740 may comprise one or more mismatch cleavage products and/or one or more indel cleavage products. Method 700 may comprise re-authenticating 732 authentication products 740 to produce re-authenticated products 740′ (not expressly shown; may be treated like products 740).

Method 700 may comprise amplifying 741 authentication products 740 (in total) or one or more species included therein. Amplifying 741 may include, for example, contacting authentication products 740 or one or more species included therein with a polymerase, for example, with a high-fidelity polymerase (e.g., Q5® High-Fidelity DNA Polymerase, which has 3′→5′ exonuclease activity that excises mismatches and cuts back 3′ overhangs), to produce amplification products 750. Method 700 may comprise transforming and/or screening amplification products 750 as described for amplification products 250 in FIG. 2. Method 700 may comprise addition (e.g., PCR addition) 771 of an additional oligonucleotide (e.g., an adapter) to one or both ends of amplification products 750 to produce tagged products 780. Adapters may comprise one or more features, including, for example, a restriction site, a bar code, a primer binding sequence, and a label (e.g., a fluorophore, an affinity tag, a ligand, or other tag).

It will be recognized that each of re-authentication 732 and amplifying 741 may be performed on a portion (e.g., an aliquot) of authentication products 740, leaving a remainder of authentication products 740 for other uses including, for example, amplification 741 and re-authentication 732, respectively. In some embodiments, method 700 may comprise denaturing 728, reannealing 729, and authentication 731 with or without additional steps shown. Method 700 may comprise, according to some embodiments, amplifying 741 and addition 771 with or without additional steps shown.

TABLE 2 illustrates nucleotide sequence information for DNA strands used to prepare example substrates for authentication. Sequence identification numbers are provided in the left column for each sequence. SEQ ID NOS: 1-12 are 60-mer oligos used to create example heteroduplex DNA. Top oligos with SEQ ID NOS: 1-4 were each paired with a copy of bottom oligo SEQ ID NO:9. Top oligos SEQ ID NOS: 5-8 were paired with one of SEQ ID NOS: 9-12 to form heteroduplexes.

TABLE 2

| Seq | Label | Sequence |
|---|---|---|
| 1 | Top-InD1 | 5′ACTCTTTCCCTACACGACGCTCTTCCGATC-GATCGGAAGAGCACACGTCTGAACTCCAG3′ |
| 2 | Top-InD2 | 5′ACTCTTTCCCTACACGACGCTCTTCCGATC--ATCGGAAGAGCACACGTCTGAACTCCAG3′ |
| 3 | Top-InD3 | 5′ACTCTTTCCCTACACGACGCTCTTCCGAT---ATCGGAAGAGCACACGTCTGAACTCCAG3′ |
| 4 | Top-InD5 | 5′ACTCTTTCCCTACACGACGCTCTTCCGAT-----CGGAAGAGCACACGTCTGAACTCCAG3′ |
| 5 | Top-A | 5′ACTCTTTCCCTACACGACGCTCTTCCGATCAGATCGGAAGAGCACACGTCTGAACTCCAG3′ |
| 6 | Top-T | 5′ACTCTTTCCCTACACGACGCTCTTCCGATCTGATCGGAAGAGCACACGTCTGAACTCCAG3′ |
| 7 | Top-G | 5′ACTCTTTCCCTACACGACGCTCTTCCGATCGGATCGGAAGAGCACACGTCTGAACTCCAG3′ |
| 8 | Top-C | 5′ACTCTTTCCCTACACGACGCTCTTCCGATCCGATCGGAAGAGCACACGTCTGAACTCCAG3′ |
| 9 | Bot-A | 3′TGAGAAAGGGATGTGCTGCGAGAAGGCTAGACTAGCCTTCTCGTGTGCAGACTTGAGGTC5′ |
| 10 | Bot-T | 3′TGAGAAAGGGATGTGCTGCGAGAAGGCTAGTCTAGCCTTCTCGTGTGCAGACTTGAGGTC5′ |
| 11 | Bot-G | 3′TGAGAAAGGGATGTGCTGCGAGAAGGCTAGGCTAGCCTTCTCGTGTGCAGACTTGAGGTC5′ |
| 12 | Bot-C | 3′TGAGAAAGGGATGTGCTGCGAGAAGGCTAGCCTAGCCTTCTCGTGTGCAGACTTGAGGTC5′ |

TABLE 3 illustrates nucleotide sequence information for a central region of a series of DNA molecules used to prepare example substrates for authentication. Sequence identification numbers are provided in the left column for each sequence. The molecules in the series have a size of 672-base (except where extended by the indicated insertions) and have sequence identity except as shown in TABLE 3.

TABLE 3

| Seq | Label | Sequence |
|---|---|---|
| 13 | 2 | TTAACTTTAAGAAGGAGATATA ACCATGAAAATCGAAGAAGGTAAAG |
| 14 | 3 | TTAACTTTAAGAAGGAGATATA CCCATGAAAATCGAAGAAGGTAAAG |
| 15 | 5 | TTAACTTTAAGAAGGAGATATA GCCATGAAAATCGAAGAAGGTAAAG |
| 16 | 8 | TTAACTTTAAGAAGGAGATATA TCCATGAAAATCGAAGAAGGTAAAG |
| 17 | 17 | TTAACTTTAAGAAGGAGATATA A ACCATGAAAATCGAAGAAGGTAAAG |
| 18 | 18 | TTAACTTTAAGAAGGAGATATA C ACCATGAAAATCGAAGAAGGTAAAG |
| 19 | 19 | TTAACTTTAAGAAGGAGATATA G ACCATGAAAATCGAAGAAGGTAAAG |
| 20 | 20 | TTAACTTTAAGAAGGAGATATA T ACCATGAAAATCGAAGAAGGTAAAG |
| 21 | 25 | TTAACTTTAAGAAGGAGATATA TG ACCATGAAAATCGAAGAAGGTAAAG |
| 22 | 28 | TTAACTTTAAGAAGGAGATATA GG ACCATGAAAATCGAAGAAGGTAAAG |
| 23 | 31 | TTAACTTTAAGAAGGAGATATA GA ACCATGAAAATCGAAGAAGGTAAAG |
| 24 | 37 | TTAACTTTAAGAAGGAGATATA CCT ACCATGAAAATCGAAGAAGGTAAAG |
| 25 | 38 | TTAACTTTAAGAAGGAGATATA TGG ACCATGAAAATCGAAGAAGGTAAAG |
| 26 | 45 | TTAACTTTAAGAAGGAGATATA ATG ACCATGAAAATCGAAGAAGGTAAAG |
| 27 | 47 | TTAACTTTAAGAAGGAGATATA CAGGT ACCATGAAAATCGAAGAAGGTAAAG |
| 28 | 48 | TTAACTTTAAGAAGGAGATATA TGTTG ACCATGAAAATCGAAGAAGGTAAAG |
| 29 | 52 | TTAACTTTAAGAAGGAGATATACCCCGGTCTCACCATGAAAATCGAAGAAGGTAAAG |
| 30 | 54 | TTAACTTTAAGAAGGAGATATACTCTGGCTACACCATGAAAATCGAAGAAGGTAAAG |

### EXAMPLES

Some embodiments may be illustrated by one or more of the examples provided herein.

### Example 1: DNA Authentication During Screening Product Creation

In many gene synthesis workflows, users obtain DNA for assembly by purchasing synthesized dsDNA or by preparing amplicons of overlapping oligonucleotides. Often, this DNA has errors that may be incorporated into the full length DNA sequence and amplified in subsequent PCR amplifications. Compositions and methods of the present disclosure may be used to remove errors (e.g., mismatches and indels) in DNA prior to or concurrent with assembly.

dsDNA may be denatured and annealed to allow heteroduplex formation between source dsDNA with errors and source dsDNA without errors. Prior to heteroduplex formation, dsDNA may be cleaned up, for example, using a spin column (e.g. Monarch® PCR & DNA Cleanup Kit (5 μg; NEB #T1030)). Cleaned up DNA samples may be eluted in a small volume (e.g. 12 μl) and DNA concentrations determined for each.

Annealing reactions may be performed, for example, with a DNA concentration of around 40 ng/μl and may be prepared with 800 ng dsDNA, 4 μl Annealing Buffer (100 mM Tris-HCl 8.0, 500 mM NaCl and 2.5 M Betaine, NEB #B2831-5X), and sufficient nuclease-free water to bring the reaction mixture to 20 μl.

A thermocycler may be used to denature and anneal the sample, forming heteroduplex ds DNA, as described in TABLE 4.

TABLE 4
Annealing Reaction Conditions

| CYCLE STEP | TEMP | | RAMP RATE | TIME |
|---|---|---|---|---|
| Initial Denaturation | 95° | C. | | 5 minutes |
| Annealing | 95-85° | C. | −2° C./second | |
| | 85-25° | C. | −0.1° C./second | |
| Hold | 4° | C. | | |

Where a starting dsDNA population comprises one or more errors, annealed dsDNA is expected to contain both (a) heteroduplex dsDNA comprising duplexes not found in the starting population and/or comprising at least one error, and (b) homoduplex dsDNA comprising duplexes like those in the starting population. Heteroduplexes may be substrates for compositions of the disclosure (e.g., T4EndoVII/EndoMS compositions). An annealed population (e.g., comprising a mixture of homoduplex and heteroduplex DNA molecules) may be contacted with an endonuclease composition (e.g., a T4Endo VII/EndoMS composition) under conditions that allow the endonucleases to cleave dsDNA at mismatches and indels.

The contacting step and endonuclease reaction may be set up on ice with the components described in TABLE 5. The 10× Reaction Buffer may include 100 mM Tris-HCl 8.0, 100 mM MgCl₂ and 1 mg/mL rAlbumin (NEB #B2832-10X).

TABLE 5
Endonuclease Reaction Components

| REAGENT | AMOUNT |
|---|---|
| Heteroduplex DNA (200 ng) | 5 μl |
| 10X Reaction Buffer | 2 μl |
| Nuclease-free water | 12 μl |
| T4EndoVII/EndoMS | 1 μl |
| Total | 20 μl |

This endonuclease reaction mixture may be incubated at 42° C. for 60 min, following which another 1.7 μl 150 mM EDTA is added and the mixture is heated at 95° C. for 5 min. The resulting authentication products may be stored at −20° C., if desired.

Authentication products may be amplified to increase the percentage of error-corrected clones. Amplification may include in two steps. In Step I the authentication products is amplified in the presence of a DNA polymerase, such as Q5® DNA polymerase but without oligonucleotide primers. In Step II, the reaction product from Step I is amplified in the presence of a DNA polymerase and oligonucleotide primers created to amplify and enrich the full size gene or other DNA sequence of interest.

In some embodiments, Step I of the amplification reaction may be prepared as described in TABLE 6. In other embodiments, it may be prepared using another error-correcting DNA polymerase with appropriate reaction buffers.

TABLE 6
Amplification Reaction Step I Components

| REAGENT | AMOUNT |
|---|---|
| authentication products | 2 μl |
| Q5 ® Hot Start High-Fidelity 2x Master Mix | 5 μl |
| Nuclease-free water | 3 μl |
| Total | 10 μl |

The mixture may be processed in a thermocycler to amplify the error-corrected DNA fragments as set forth in TABLE 7.

TABLE 7
Amplification Reaction Step I Conditions

| CYCLE STEP | TEMP | RAMP RATE | TIME | CYCLES |
|---|---|---|---|---|
| Initial Denaturation | 95° C. | | 5 minutes | |
| Annealing | 95-72° C. | 0.1° C./second | | |
| Hold | 72° C. | | 10 min | |
| Denaturation | 98° C. | | 10 seconds | 23 |
| Annealing | 64° C.* | | 10 seconds | |
| Extension (for | 72° C. | | 30-50 | |
| 500-1000 bp) | | | seconds | |
| Final Extension | 72° C. | | 3 minutes | |

In some embodiments of Step II, two nearly identical reactions (Tube A and Tube B) are created to amplify and enrich the full size gene or other DNA of interest. The first reaction (Tube A) uses 2 μl of the error-corrected DNA fragments from Step I as the template. The second reaction (Tube B) uses 2 μl of products from the first reaction (Tube A) as the template to ensure appropriate amplification. In other embodiments, only a single reaction (Tube A) is prepared.

A PCR reaction mix may be prepared with a volume of 50 μl and with 0.5 μM of Forward/Reverse primers created to amplify and enrich the full size gene of interest. 25 μl of the PCR reaction mix is transferred to Tubes A and B. 2 μl of template from Step I is added to Tube A and mixed properly. In some embodiments, 2 μl of the Tube A mix to is transferred to Tube B. This will create 2 PCR reactions with 2 μl (Tube A) and 0.16 μl (Tube B) of template from Step I. The composition of the reaction mix for Tubes A and B are summarized in TABLE 8.

TABLE 8
Amplification Reaction Step II Components

| | | | Rxn FINAL |
|---|---|---|---|
| REAGENT | Tube A | Tube B | CONCENTRATION |
| Q5 ® Hot Start High- | 12.5 μl | 12.5 μl | 1X |
| Fidelity 2X Master | | | |
| Mix | | | |
| 10 μM Forward | 1.25 μl | 1.25 μl | 0.5 μM |
| Primer | | | |
| 10 μM Reverse | 1.25 μl | 1.25 μl | 0.5 μM |
| Primer | | | |
| Nuclease-free water | 10 μl | 10 μl | |
| Template DNA | 2 μl | Add 2 μl of | |
| | | Tube A mix | |

The mixtures of Tube A and Tube B may be processed in a thermocycler to amplify the error-corrected pool as summarized in TABLE 9.

TABLE 9
Amplification Reaction Step II Conditions

| CYCLE STEP | TEMP | TIME | CYCLES |
|---|---|---|---|
| Initial Denaturation | 98° C. | 2 minutes | |
| Denaturation | 98° C. | 10 seconds | 23 |
| Annealing | 64° C.* | 10 seconds | |
| Extension (for 500-1000 bp) | 72° C. | 30-50 seconds | |
| Final Extension | 72° C. | 5 minutes | |
| Hold 4-10° C. | | | |

The purity of PCR products from Tubes A and B may be assessed and the PCR product with higher purity selected as the amplification product. For example, a portion of the PCR product, such as 10%, may be run on an agarose gel. Other methods such as Bioanalyzer or TapeStation may also be used.

The amplification product may undergo a transformation step in which DNA is ligated or assembled into a destination vector of choice that is then transformed into competent cells.

Typically, traditional Restriction Enzyme digestion and ligation, NEBuilder HiFi DNA Assembly, or Golden Gate Assembly methods using NEBridge reagents are used for vector assembly.

Amplification products may be cleaned up by spin column prior to transformation, for example, to improve quantitation accuracy and/or to remove potential inhibitors of enzymes used in future steps. In other embodiments, assembled products may be used directly without cleanup, but transformation efficiency may be reduced.

Assembled DNA may then be transformed into competent bacteria, such as E. coli (e.g. NEB 5-alpha or NEB 10-beta) and propagated on rich agar plates with appropriate antibiotic selection.

The propagated bacteria may undergo a screening step in which colonies are picked and analyzed for presence of correct clones. In some embodiments, colonies are analyzed directly by colony PCR with appropriate primers followed by agarose gel electrophoresis and Sanger sequencing of the amplicon. In other embodiments, colonies are analyzed indirectly by analysis of miniprep plasmid DNA after overnight culture by restriction enzyme digest or Sanger DNA sequencing. The resulting screening products may include colonies or vectors that encompass the correct DNA sequence for the synthesized gene or other DNA of interest.

### Example 2: DNA Fragment Analysis

Heterogeneous cell populations created by genome editing techniques (CRISPR, TALEN, ZFN, etc.) may be screened using authentication methods of the present disclosure to identify DNA fragments containing mismatches and/or indels. Cleaved dsDNA substrates in authentication products may be identified using an agarose gel or Bioanalyzer. The proportion of uncut to cut DNA fragments may be determined to provide an estimate of the efficiency of the genome editing event. By recognizing a more comprehensive set of structures, compared to T4EndoVII or EndoMS alone, use of T4Endo VII/EndoMS compositions of the present disclosure may improve the accuracy of the DNA fragment analysis.

The heterogeneity in a population of DNA molecules may be predicted on theoretical grounds or estimated empirically. For example, two analytical methods (here designated “Method 1” and “Method 2”) may be used to estimate the heterogeneity within a source dsDNA pool generated by PCR amplification of an edited target region. In Method 1, DNA is amplified from edited cells. In Method 2, DNA is amplified from both edited cells and wild type cells. Including DNA from unedited cells may serve as a useful control and improve the accuracy of calculations, for example, where there is a dominant mutation (previously identified or suspected).

DNA from edited cells and, optionally DNA from wild type cells, may undergo an amplification step. If Method 1 is followed, amplification of only edited (Rxn A) populations is sufficient.

A PCR reaction mixture may be prepared by setting up one (for Method 1) or two (for Method 2) 25 μL PCR reactions that each include up to 500 ng of genomic DNA as templates. The reactions may be prepared at room temperature. The composition of the PCR reaction mixture(s) is described in TABLE 10. Reaction A is the experimental reaction with edited genomic DNA as template. Reaction B is the control reaction using gDNA from non-edited (wild type) cells.

TABLE 10
Amplification Reaction Components

| | Rxn A: | Rxn B: | |
|---|---|---|---|
| | 25 μL | 25 μL | |
| | reaction | reaction | RXN FINAL |
| | with | with | CONCEN- |
| REAGENT | Edited cells | WT cells | TRATION |
| Q5 ® Hot Start High- | 12.5 μL | 12.5 L | 1X |
| Fidelity 2X Master Mix | | | |
| 10 μM Forward Primer | 1.25 μL | 1.25 μL | 0.5 μM |
| 10 μM Reverse Primer | 1.25 μL | 1.25 μL | 0.5 μM |
| Template DNA (edited | variable | | 0.5-500 ng genomic |
| genome) | | | DNA |
| Template DNA (WT | | variable | 0.5-500 ng genomic |
| genome) | | | DNA |
| Nuclease-free water | to 25 μL | to 25 μL | |

Primers may be designed to produce amplicons around 700 bp with anticipated sizes of cleaved dsDNA substrate around 450 and 250 bp.

The mixtures of Reaction A and Reaction B may be processed in a thermocycler to amplify the template DNA as summarized in TABLE 11.

TABLE 11
Amplification Reaction Conditions

| CYCLE STEP | TEMP | TIME | CYCLES |
|---|---|---|---|
| Initial Denaturation | 98° C. | 2 minutes | |
| Denaturation | 98° C. | 10 seconds | 35 |
| Annealing | 50-72° C.* | 5 seconds | |
| Extension (for 500-700 bp) | 72° C. | 30 seconds | |
| Final Extension | 72° C. | 2 minutes | |
| Hold 4-10° C. | | | |

Appropriate annealing temperatures may be calculated.

Amplified source dsDNA may be denatured and annealed to allow heteroduplex formation between DNA fragments with and without errors. Rapid qualitative analysis may comprise reannealing unpurified PCR amplicons followed by contact with the T4Endo VII/EndoMS compositions of the present disclosure. Resulting DNA fragments may be analyzed by agarose gel electrophoresis.

Amplification products may be purified prior to fragment analysis, for example, if genome editing efficiency is to be calculated. Amplified dsDNA purification may comprise enzymatic treatment and/or spin column purification.

### Method 1

Method 1 uses PCR amplicons from the genomes of edited cells, for example, as illustrated in the upper portions of FIG. 6A and FIG. 6B. Method 1 does not require PCR amplicons from the genome of unedited cells. To prepare DNA for accurate quantitation, PCR reaction products may be cleaned up by an enzymatic method or a column method prior to preparation of heteroduplex DNA.

For enzymatic cleanup, reactions may be prepared as described in TABLE 12.

TABLE 12
Enzymatic Cleanup Reaction Components

| REAGENT | ANNEALING REACTION | |
|---|---|---|
| PCR Reaction (unpurified) | 18 | μL |
| Thermolabile ExoI (M0568) | 1 | μL |
| Quick-CIP (M0525) | 1 | ul |
| Total | 20 | ul |

Reaction tubes may be briefly spun down and incubated at 37° C. for 4 min followed by 80° C. for 1 min.

Column cleanup may be performed, for example, by using the Monarch® PCR & DNA Cleanup Kit (5 μg) (NEB #T1030) with elution volume of 12 μl. dsDNA concentration may be measured. Annealing reactions may be prepared as described in TABLE 13.

TABLE 13
Annealing Reaction Components

| REAGENT | ANNEALING REACTION |
|---|---|
| Cleanup PCR amplicons (400 ng) | 1-16 μL |
| 5x annealing buffer | 4 μL |
| Nuclease-free water | to 20 μl |

Heteroduplex dsDNA substrates may be formed in a thermocycler using the program described in TABLE 14.

TABLE 14
Annealing Reaction Conditions

| CYCLE STEP | TEMP | RAMP RATE | TIME |
|---|---|---|---|
| Initial Denaturation | 95° C. | | 3 minutes |
| Annealing | 95-85° C. | 2° C./second | |
| | 85-25° C. | −0.1° C./second | |
| Hold | 4° C. | | |

Alternatively, a sample may be heated to 95° C. for 10 minutes and then allowed to cool slowly to room temperature.

Heteroduplex dsDNA substrates may be contacted with a composition of the present disclosure (e.g., a T4Endo VII/EndoMS composition) to cleave DNA at mismatches and indels. Endonuclease reactions may be prepared as described in TABLE 15.

TABLE 15
Endonuclease Reaction Components

| | Enzymatic cleanup | | | | Column purified | | |
|---|---|---|---|---|---|---|---|
| | amplicons | | | | amplicons | | |
| | | | Negative | | | | Negative |
| REAGENT | REACTION | | Control | | REACTION | | Control |
| Annealed PCR | 1-12 | μL* | 1-12 | ul* | 10 | μL | 10 ul |
| amplicons (~200 ng) | | | | | | | |
| 10x Reaction Buffer | 2 | ul | 2 | ul | 2 | ul | 2 ul |
| water | 16-5 | ul | 17-6 | ul | 7 | ul | 8 ul |
| Authenticase | 1 | μL | 0 | ul | 1 | μL | 0 ul |
| Total | 20 | ul | 20 | ul | 20 | ul | 20 ul |

Endonuclease reaction conditions are optimized for up to 6 μL of the unpurified enzyme-treated Q5® Master Mix PCR reaction product or 12 μl of unpurified OneTaq PCR reaction product containing up to 200 ng of amplified DNA. Increased amounts of PCR reaction product and/or DNA may lead to inaccurate estimates of editing efficiencies.

Reaction tubes may be mixed well and then spun briefly. Each tube may be incubated at 42° C. for 15 minutes. Reactions may be stopped with 1.7 μl of 150 mM EDTA. Reaction products may be analyzed (e.g., by DNA fragment analysis) directly or tubes may be stored at −20° C.

### Method 2

Method 2 uses PCR amplicons from the genomes of both edited cells and wild type cells, for example, as illustrated in the lower portions of FIG. 6A and FIG. 6B. To prepare DNA for accurate quantitation, PCR reaction products may be cleaned up by an enzymatic method or a column method prior to preparation of heteroduplex dsDNA.

For enzymatic cleanup, reactions may be prepared as described in TABLE 16.

TABLE 16
Enzymatic Cleanup Reaction Components

| REAGENT | REACTION A | REACTION B |
|---|---|---|
| PCR amplicons from | 18 μl | |
| edited genome | | |
| PCR amplicons from | | 18 μl |
| WT genome | | |
| TL-ExoI (M0568) | 1 μL | 1 μL |
| Quick-CIP (M0525) | 1 ul | 1 μl |
| water | to 20 μl | to 20 μl |

Reaction tubes may be spun down briefly and incubated at 37° C. for 4 min followed by 80° C. for 1 min. Column cleanup may be performed, for example, by using the Monarch® PCR & DNA Cleanup Kit (5 μg) (NEB #T1030) with elution volume of 12 μl. dsDNA concentration may be measured. Annealing reactions to form heteroduplex DNA may be performed by mixing 200 ng of reaction A (from edited gDNA template) and 200 ng of reaction B (from WT gDNA template) and then incubating in a thermocycler using the program described in TABLE 17.

TABLE 17
Annealing Reaction Conditions

| CYCLE STEP | TEMP | RAMP RATE | TIME |
|---|---|---|---|
| Initial Denaturation | 95° C. | | 3 minutes |
| Annealing | 95-85° C. | 2° C./second | |
| | 85-25° C. | −0.1° C./second | |
| Hold | 4° C. | | |

Alternatively, samples may be heated to 95° C. for 10 minutes and then allowed to cool slowly to room temperature.

Heteroduplex dsDNA substrates may be contacted with a composition of the present disclosure (e.g., a T4Endo VII/EndoMS composition) to cleave DNA at mismatches and indels. Endonuclease reaction may be prepared as described in TABLE 18.

TABLE 18
Endonuclease Reaction Components

| | Exol and CIP | | Column | |
|---|---|---|---|---|
| | Treated Amplicons | | Purified | |
| | 20 μL | | Amplicons | |
| REAGENT | REACTION | Control | | Control |
| Annealed PCR | (X + Y)/2 μL* | — | 10 μL | — |
| Product (~200 ng) | | | | |
| Anneal PCR Product | — | (X + Y)/2 μl* | — | 10 ul |
| (~200 ng) | | | | |
| 10x Reaction Buffer | 2 μl | 2 μl | 2 μl | 2 μl |
| water | 17-(X + Y)/2 μl | 18-(X + Y)/2 μl | 7 μl | 8 μl |
| Authenticase | 1 μL | 0 μl | 1 μL | 0 μl |
| Total | 20 μl | 20 μl | 20 μl | 20 μl |

Endonuclease reaction conditions are optimized for up to 6 μL of the unpurified enzyme-treated Q5® Master Mix PCR reaction product or 12 μl of unpurified OneTaq PCR reaction product containing up to 200 ng of amplified DNA. Increased amounts of PCR reaction products and/or DNA may lead to inaccurate estimates of editing efficiencies.

Reaction tubes may be mixed well and then spun briefly. Each tube may be incubated at 42° C. for 15 minutes. Reactions may be stopped with 1.7 μl of 150 mM EDTA. Reaction products may be analyzed (e.g., by DNA fragment analysis) directly or the tubes may be stored at −20° C.

### Authentication Product Analysis

Authentication products from Method 1 or Method 2 may undergo DNA fragment analysis to estimate the efficiency of genetic modification.

Such analysis may be performed using gel electrophoresis. 4 μL of Gel Loading Dye, Purple (6X, NEB #B7024) may be added to the reaction product and run on a 2% agarose gel stained with ethidium bromide. An appropriate DNA size marker may be run alongside the sample for reference.

Alternatively, authentication products samples may be analyzed using a fragment analyzer (e.g. Agilent Bioanalyzer or Advanced Analytical Technologies, Inc (AATI) Fragment Analyzer). For example, fragment analyzer analysis may comprise diluting 2 μL of enzyme-treated sample in 8 μL of water and analyzing 1 μL of the diluted mixture on a high sensitivity Agilent DNA chip. This allows detection of populations with DNA errors down to 1 out of 80 copies based on 690 bp PCR amplicon design with cleaved product sizes of 450 bp and 240 bp. For the AATI Fragment Analyzer, 2 μL of the reaction product may be used with the Standard Sensitivity NGS Fragment Analysis Kit (AATI Cat #DNF-473) in accordance with the manufacturer's instructions. Example Bioanalyzer results are shown FIG. 5 and TABLE 1 in connection with method 500.

### Theoretical Error Estimation

In a dsDNA population, the total number of molecules comprising an error (“E”) may be expressed as a simple sum of the number of molecules having each error subtype (assuming there is only one error per molecule) of n total subtypes:

$${E}_{Duplex} = {}^{S 1} {E}_{Duplex} + {}^{S 2} {E}_{Duplex} + {}^{S 3} {E}_{Duplex} + … + {}^{Sn} {E}_{Duplex}$$

The total number of duplex molecules in a population (“Total Duplex” or “T_{Duplex}”) may be expressed as the sum of molecules comprising an error (“E_{Duplex}”) and molecules that are error-free (“EF_{Duplex}”):

$${T}_{Duplex} = {EF}_{Duplex} + {E}_{Duplex}$$

The mole percentage of heteroduplex DNA (e.g., formed upon melting and annealing as disclosed herein) in a dsDNA population may be expressed as:

$$\% of heteroduplex = \frac{moles of P 1}{( moles of P 1 + moles of uncut dsDNA )}$$

or this may be expressed as:

$$\% of heteroduplex = \frac{Number of subtype 1 mutation * [ Number of ( WT + subtype 2 , 3 … mutation ) ] + Number of subtype 2 mutation * [ Number of ( WT + subtype 1 , 3 … mutation ] + Number of subtype 3 mutation * [ Number of ( WT + subtype 1 , 2 … mutations ]}{Total number of duplex combination}$$

### Empirical Error Estimation

As disclosed in connection with method 500, the mole percentage of heteroduplex molecules in complex population may be given by Formula I:

$$\% heteroduplex = [ P 1 / ( P 1 + S ) ] , \qquad ( I )$$

wherein P1 is the number (e.g. moles) of one size of cleaved DNA substrate fragment and S is the number (e.g. moles) of the uncleaved DNA fragments (i.e. the error-free DNA fragments). Estimated mole percentages of dsDNA comprising errors may be given by Formula II or Formula III as follows:

$$Method 1 \% Error = 100 \times [ 1 - ( \sqrt{1 - \% Heteroduplex} ) ] \qquad ( II )$$

$$Method 2 \% Error = \{ 100 \times [ 1 - \sqrt{1 - fraction cleaved} ] \} \times 2 \qquad ( III )$$

When calculating % modification for reactions with the control template where the starting material is known, the equation (100×fraction cleaved) may be used, where fraction cleaved (also referred to as % or proportion heteroduplex)=molarity of cleaved DNA substrate/(molarity of cleaved DNA substrate+molarity of uncut DNA fragments). Using the TABLE 1 data that obtained in connection with the methods disclosed in this example, the % Error is given by:

$$\begin{matrix}\% Error = 100 \times [ 1 - ( \sqrt{1 - P 1 / ( P 2 + S )} ] \\ = 100 \times [ 1 - ( \sqrt{1 - 1728 / ( 1728 + 1436 )} ] \\ = 32.6 \%\end{matrix}$$

In some embodiments, the % heteroduplex dsDNA is then used to calculate an error rate in the dsDNA source (which may be referred to as the percent modification) in the authentication products dsDNA. The percent error may be calculated by formula II above.

### Example 3: Assembly and Analysis of MBD Gene and lacZ-GFP Constructs

The MBD gene and a lacZ-GFP construct were assembled from commercially synthesized oligonucleotides. For the MBD gene (645 bp), 16 oligonucleotides (MBD-1 to MBD-16) were used as templates and MBD-1 and MBD-16 were used as forward and reverse primers in the assembly PCR reaction. For lacZ-GFP gene (967bbp), 24 oligonucleotides (ozGFPF1-12 and ozGFPR1-12) were used as templates and lacZ-GFP_F and lacZ-GFP R3 were used as forward and reverse primers in the assembly PCR reaction.

500 mol of each oligo were used as templates in a 50 μl PCR reaction with 36 amplification cycles. Amplicons were cleaned up in a spin column. Amplicons were divided into three pools: the first was left uncorrected, the second was contacted with a T4EndoVII/EndoMS composition of the present disclosure as described in Example 1, and the third was corrected by CORRECTASE (ThermoFisher) according to the manufacturer's instructions.

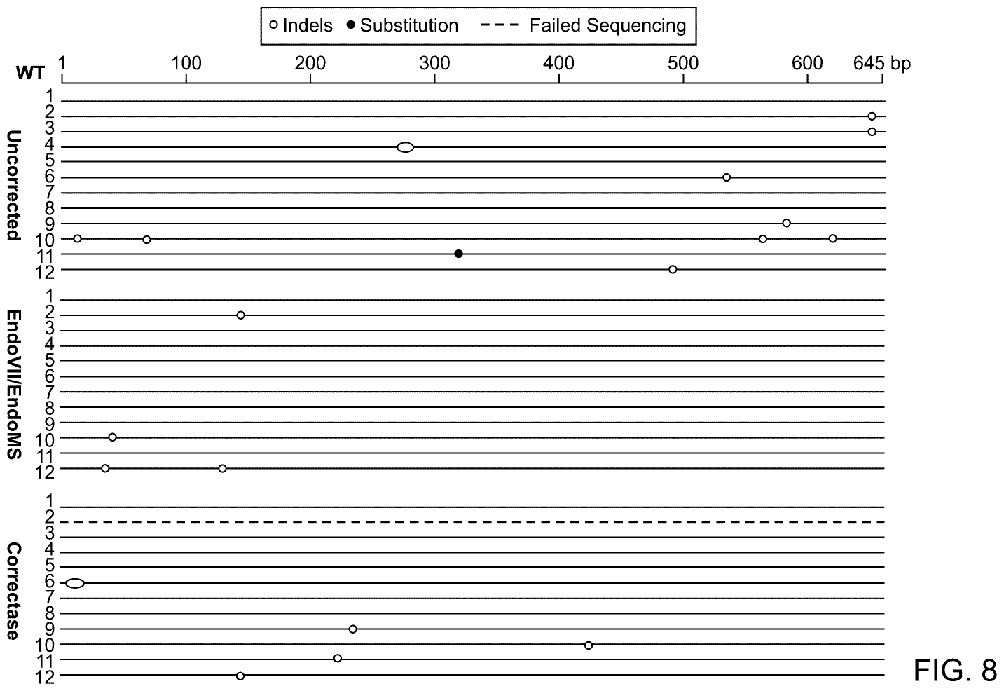

Uncorrected MDB gene amplicons and amplicons authenticated by T4Endo VII/EndoMS or corrected by CORRECTASE were cloned into linear pUC19 vectors which were amplified by PCR using MBD-pUC19F and MBD-pUC19R primers. PCR fragments and vector were assembled using NEBuilder HiFi DNA assembly master mix followed by transformation into DH5-alpha competent cells. Twelve colonies from each pool (uncorrected, contacted with T4Endo VII/EndoMS and corrected with CORRECTASE) were picked. Plasmids from each colony were purified and sequenced by the Sanger DNA sequencing method. Results are indicated in FIG. 8 and TABLE 19. TABLE 19 also provides results from a similar assay in which the MDB amplicons were contacted with T4Endo VII/EndoMS for a shortened period of 30 minutes. Error rates in TABLE 19 were determined as the total number of errors divided by the total number of bases sequenced.

TABLE 19
Sequencing Resμlts of Colony-PCR/
miniprep DNA from Assembled MBD gene

| Treatment Protocol | Error Rate | % of Correct Sequences |
|---|---|---|
| None | 12 errors/7740 bases or | 33% (4/12) |
| | 1 error/~645 bases | |
| T4Endo VII/EndoMS | 6 errors/7740 bases or | 58% (7/12) |
| (30 min treatment) | 1 error/~1290 bases | |
| T4Endo VII/EndoMS | 4 errors/7740 bases or | 75% (9/12) |
| (60 min treatment) | 1 error/~1935 bases | |
| CORRECTASE | 5 errors/7095 bases or | 50% (6/12) |
| | 1 error/~1419 bases | |

Uncorrected lacZ-GFP amplicons, lacZ-GFP amplicons contacted with T4Endo VII/EndoMS, or amplicons corrected by CORRECTASE were cloned into linear pUC19 vectors which are amplified by ozGFP-pUC19F/ozGFP-pUC19R primers. PCR fragments and vectors were assembled using NEBuilder HiFi DNA assembly master mix followed by transformation into DH5-alpha competent cells. Twelve colonies from each plate (uncorrected, contacted with T4Endo VII/EndoMS (15, 30, and 60 min) and treated with CORRECTASE were picked, and plasmids were purified and sequenced by Sanger DNA sequencing. As a separate verification, colonies containing correct assembled constructs may be visualized under UV lamp and the percent of fluorescence colonies calculated. The results of the above experiment are summarized in TABLE 20 below. Error rates were determined as the total number of errors (e.g., counting as a single error both a single base error and a consecutive series of base errors) divided by the total number of bases sequenced.

TABLE 20
Sequencing and Fluorescence Resμlts of miniprep
DNA from Assembled lacZ-GFP

| | | % of | |
|---|---|---|---|
| | | Correct | % of |
| | | Sequenced | Fluorescent |
| Treatment Protocol | Error Rate | Clones | Colonies |
| None | 10 errors/4835 bases or | 17% | 48% |
| | 1 error/~484 bases | (2/12) | (19/40) |
| T4EndoVII/EndoMS | N/A | N/A | 58% |
| (15 min treatment) | | | (71/123) |
| T4EndoVII/EndoMS | 1 errors/9153 bases or | 67% | 74% |
| (30 min treatment) | 1 error/~9153 bases | (8/12) | (53/72) |
| T4EndoVII/EndoMS | 3 errors/10637 bases or | 67% | 77% |
| (60 min treatment) | 1 error/~3546 bases | (8/12) | (81/105) |
| CORRECTASE | 3 errors/10670 bases or | 75% | 79% |
| | 1 error/~3556 bases | (9/12) | (122/155) |

### Example 4:60-mer Oligonucleotide Assay

60-mer oligonucleotides containing indels or mismatches were synthesized. Sequences for the top oligonucleotides of 55-60 bases and for the bottom oligonucleotides of 60 baser are shown in TABLE 2. Each pair of top and bottom oligonucleotides were combined and annealed to generate eight different heteroduplex dsDNA substrates. 1 pmol of each heteroduplex dsDNA substrate was then contacted with T4EndoVII/EndoMS for 30 min at 42° C. in 20 μl of authentication reaction buffer (to generate results shown in FIG. 9A) or with 1 μl of T7 endonuclease I for 30 min at 37° C. in a 20 μl 1× NEBuffer r2.1 reaction buffer (to generate results shown in FIG. 9B). The digested dsDNA are then analyzed on an E-gel (EX 4% agarose) from Thermo Fisher Scientific. 16 combinations of single-base mismatch substrates were tested as well as 1, 2, 3 and 5 bp InDel substrates. The results shown in FIG. 9A and FIG. 9B indicate that T4EndoVII/EndoMS showed improvement over existing commercial T7 Endonuclease I on those heteroduplex substrates labelled with stars (T/T, T/G, G/G and G/T mismatches).

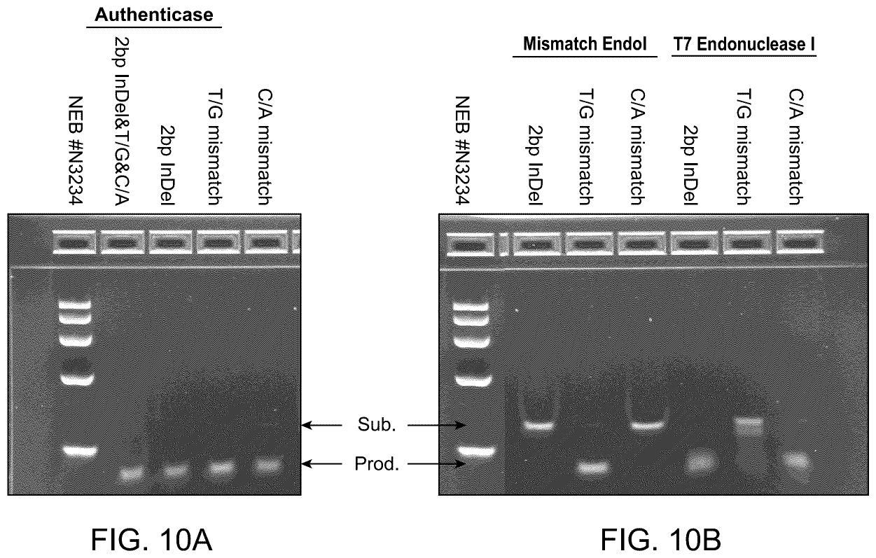

In a first assay, 1 μL of T4EndoVII/EndoMS was used in a 20 μL reaction containing 1× authentication reaction buffer to form a mixture of 0.33 pmol of a combination of 60-mer heteroduplex dsDNA substrates with A/C mismatches, T/G mismatches, and 2 bp indels. After incubation at 42° C. for 30 min, >90% of the dsDNA substrate was cleaved to 30-mers as determined by analysis on 4% agarose E-gel with results shown in FIG. 10A. Comparative assays using T7 endonuclease I and Mismatch Endonuclease I alone were also performed and results are shown in FIG. 10B. The results indicate the T4EndoVII/EndoMS cleaved heterogenous dsDNA at error locations more effectively than either single comparative enzyme and exhibited a broader range of error recognition.

Furthermore, because the sample contained a mix of dsDNA substrates with different errors, the results demonstrate that T4EndoVII/EndoMS functions effectively in a heterogenous heteroduplex dsDNA substrate population.

### Example 5: Mismatch Cleavage Assay on Long Homogeneous Heteroduplex DNA

A series of plasmids were engineered to include a 672 bp region comprising the sequences shown in TABLE 3. Capitalizing on differences in these disclosed sequences, homogeneous heteroduplex DNA substrates were prepared from PCR amplicons according to method 400 shown in FIG. 4. Four plasmid constructs (comprising SEQ ID NOS: 13-16) containing a specific mutation at the same locus and differentially phosphorylated primers (either forward or reverse) were used to generate eight double-stranded PCR generated fragments ˜672 bp in length (ds1-ds8). Following cleanup (Monarch PCR & DNA Cleanup Kit (NEB #T1030)), the phosphorylated strand of the double-stranded fragments (2.2 μg) was specifically degraded at 37° C. over 60 minutes using 5 units of Lambda Exonuclease to generate single-stranded oligos of either the top or bottom strand containing either A, T, C or G at the same locus. The single-stranded oligos (ss1-ss8) were then purified using the Oligonucleotide Cleanup Protocol for the Monarch PCR & DNA Cleanup Kit (NEB #T1030).

Purified single-stranded oligos containing either an A, T, C or G can then be mixed and matched to form either perfectly Watson-Crick base-paired DNA (green check marked boxes) or double-stranded DNA oligos containing a single-base mismatch. Mismatched dsDNA oligos were generated by mixing the appropriate top and bottom strands (for the mismatch to be created) and re-annealing in 1× NEBuffer 2.1, heated to 95° C., followed by cooling to room temperature, to generate the eight potential DNA mismatches (A:A, A:C, A:G, C:C, C:T, G:G, G:T, T:T).

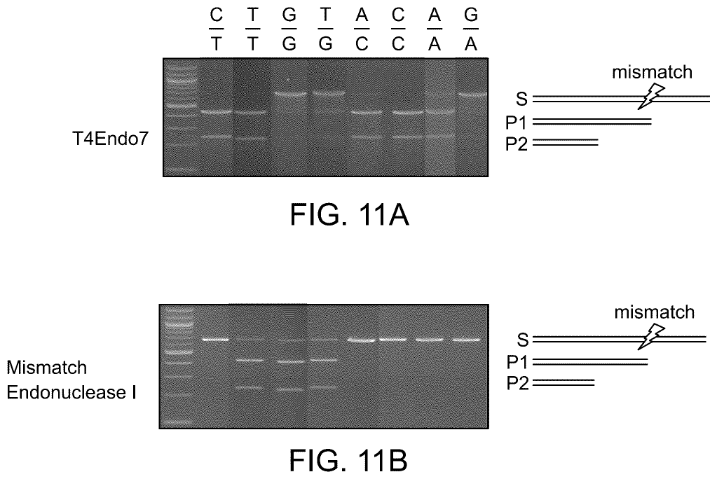

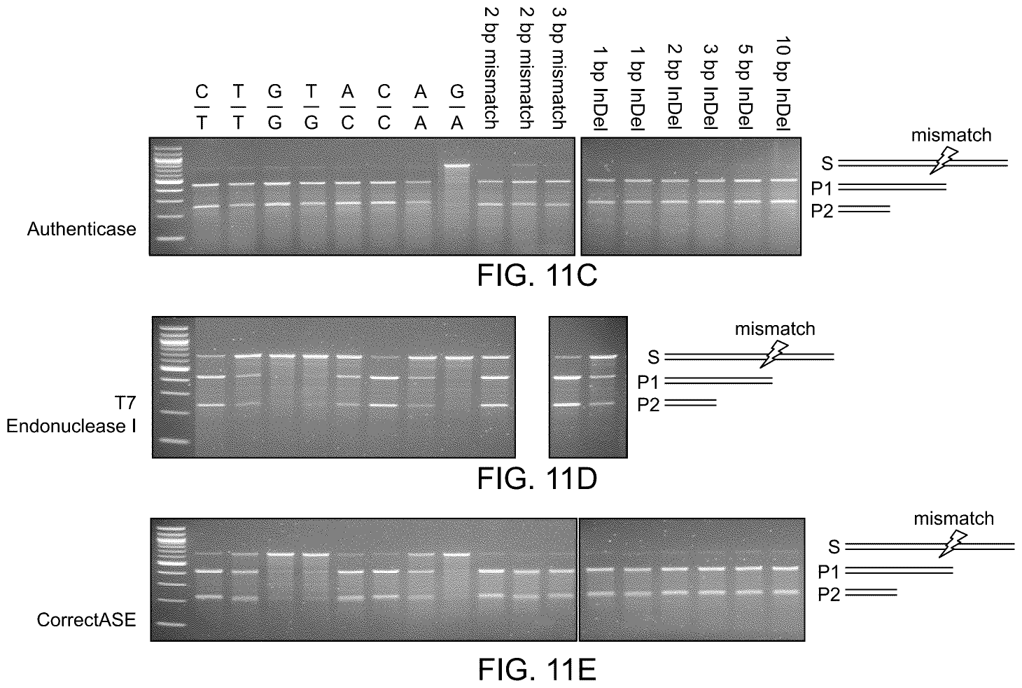

Additional homogenous heteroduplex dsDNA substrates were constructed as described in FIG. 4, some with single base mismatches, some with a 2 bp, 3 bp, or 5 bp mismatch, and some with a 1 bp, 2 bp, 3 bp, or 5 bp indel, Using template plasmids comprising SEQ ID NOS: 13-30. These substrates were digested with T4EndoVII alone, mismatch endonuclease I alone, T4EndoVII/EndoMS, T7 endonuclease I alone, or CorrectASE™ alone. The ability of these endonuclease compositions to cut specific mismatches in dsDNA was queried by incubating 1 μL of the endonuclease composition with 200 ng of mismatch containing dsDNA. Reactions with T4EndoVII/EndoMS were incubated at 42° C. for 30 minutes, reactions with CorrectASE™ were incubated at 25° C. for 60) minutes, and reactions with EndoMS alone. T4EndoVII alone, or T7 endonuclease I alone were incubated at 37° C. for 60) minutes, in each case, in a reaction buffer as recommended by the respective manufacturer for commercially available enzymes. T4EndoVII/EndoMS were incubated in NEB Buffer 2.1. Reaction products were then run on a 1.2% agarose gel and visualized with ethidium bromide staining. Results are shown in FIGS. 11A-11E. T4Endo VII/EndoMS cleaved all heteroduplex dsDNA substrates near the error, except the heteroduplex dsDNA substrate with a G/A mismatch. CorrectASE™, T4E7 alone, and T7 endonuclease I alone did not cleave heteroduplex dsDNA substrates with G/G, G/T, or T/G mismatches. The T4EVII/EndoMS composition tested displayed broader substrate specificity as compared to T4EndoVII alone or mismatch EndoI alone where initial substrates are digested into two product fragments.

### Example 6: Mismatch Cleavage Assay on Long Heterogeneous Heteroduplex DNA

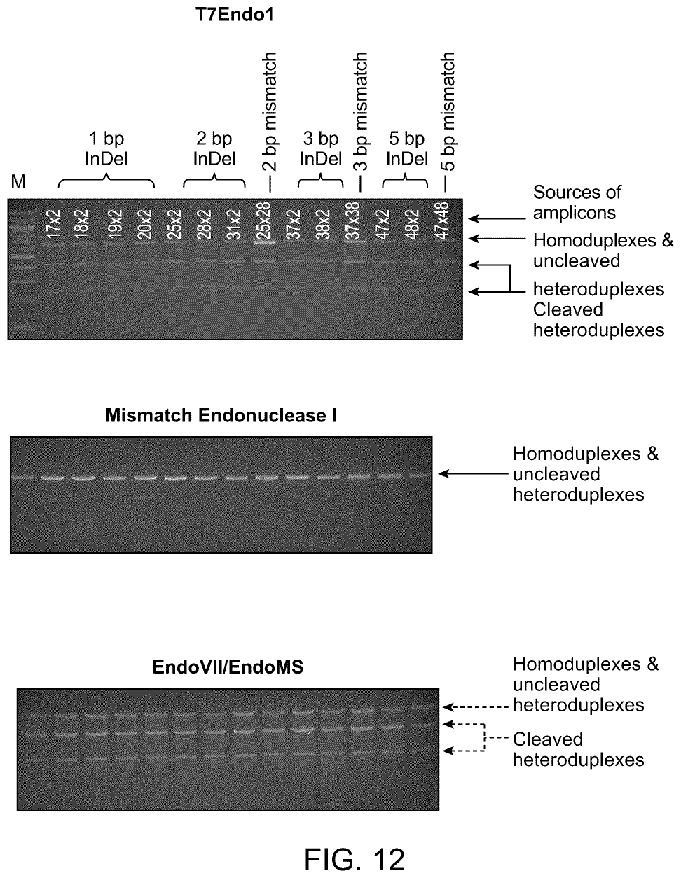

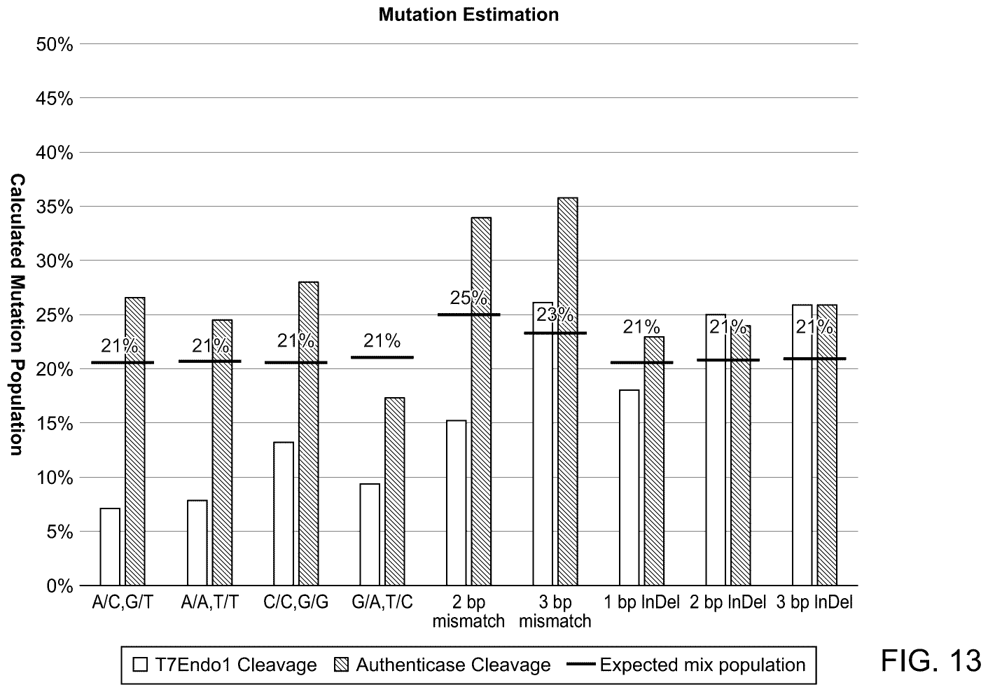

Plasmids engineered to include a 672 bp region comprising the sequences shown in TABLE 3 were Used to prepare heterogeneous heteroduplex DNA from PCR amplicons according to method 500 (FIG. 5). PCR amplicons were mixed, annealed in 1× NEBuffer 2, heated to 95° C. and cooled to room temperature. Fourteen heterogeneous heteroduplex dsDNA substrates were generated in this manner. The number on the top of gels shown in FIG. 12 indicate sources of amplicons from Table 3, e.g., 17×2 refers to using equal amount of amplicons from label #17 and label #2 RCR), resulting in dsDNA fragments containing approximately 50% homoduplex (error-free) dsDNA fragments, which are resistant to cleavage by EndoVII/EndoMS and 50% heteroduplex (errors) dsDNA fragments, which are sensitive to cleavage by EndoVII/EndoMS.

Heterogeneous heteroduplex dsDNA fragment samples containing approximately 50% heteroduplex dsDNA with a 2 bp, 3 bp, or 5 bp mismatch, or a 1 bp, 2 bp, 3 bp, or 5 bp indel along with approximately 50% corresponding homoduplex, error-free DNA fragments were prepared and used to evaluate substrate specificity of an EndoVII/EndoMS composition. T7 endonuclease I alone, or EndoMS alone. Results are shown in FIG. 12. Both EndoVII/EndoMS and T7 endonuclease I alone cleaved all heteroduplex dsDNA substrates, while not cleaving homoduplex dsDNA fragments. EndoMS alone was unable to cleave most of the dsDNA fragments, regardless of whether an error was present. These results are consistent with the T4Endo VII/EndoMS composition tested complimenting the otherwise deficient activity of mismatch endo I in different types of indel substrates and >1 bp mismatch substrates.

### Example 7: DNA Fragment Analysis Using DNA Authentication

dsDNA fragment samples containing known ratios of heteroduplex dsDNA substrate (designated S) and error-free dsDNA fragments (designated WT). The samples contained a heterogenous mixture of heteroduplex dsDNA substrates with different errors. Each type of dsDNA substrate with a different error is designated S1, S2 . . . Sx. Theoretical estimations of the proportion of heteroduplex dsDNA was calculated as follows:

$$\% heteroduplex dsDNA = [ ( S 1 mol ( WT mol + S 2 mol + … Sx mol ) ) + ( S 2 mol ( WT mol + S 1 mol + … Sx mol ) + … ( Sx mol ( WTmol + S 1 mol + S 2 mol + … Sx - 1 mol ) ) ] / [ WT mol + S 1 mol + S 2 mol + … Sx mol ]$$

Theoretical estimates for each sample are provided in FIG. 14 (black bars).

The samples were then contacted with either T4EndoVII/EndoMS or T7 endonuclease I and the products were analyzed using a Bioanalyzer and % heteroduplex dsDNA, as measured, was calculated as provided in method 300 above and results are provided in FIG. 14. T4EndoVII/EndoMS consistently yielded measured % heteroduplex dsDNA that more closely matched the results that should theoretically have been obtained.

What is claimed is:

1. An enzyme composition comprising a T4EndoVII endonuclease and a mismatch endonuclease, wherein
(a) the composition is cell-free,
(b) the composition has a temperature of 30° C. to 45° C.,
(c) the composition comprises ≥1 unit of T4EndoVII per μL and ≥1 unit of μL of the mismatch endonuclease,
(d) the composition further comprises a buffering agent, or
(e) the mismatch endonuclease is EndoMS.

2. An enzyme composition according to claim 1, further comprising a polymerase, a base editor, a restriction enzyme, NTPs, or any combinations thereof.

3. An enzyme composition of claim 1, wherein the composition is in a dried form, a freeze dried form, a lyophilized form, a crystalline form, an aqueous form, a liquid form, or an immobilized form.

4. An enzyme composition according to claim 1, further comprising dsDNA molecules comprising heteroduplex dsDNA comprising at least one error and homoduplex dsDNA.

5. An enzyme composition according to claim 4, wherein the at least one error comprises a mismatch or an indel.

6. An enzyme composition according to claim 4, wherein the dsDNA molecules comprise heteroduplex dsDNA substrates comprising different errors.

7. An enzyme composition according to claim 6, wherein the different errors comprise both mismatches and indels.

8. An enzyme composition according to claim 4, wherein the homoduplex dsDNA is identical to a reference DNA in length and sequence.

9. A kit comprising:
(a) a T4EndoVII endonuclease;
(b) a mismatch endonuclease; and
(c) a buffer or buffering agent, an additive, instructions for use, or any combinations thereof.

10. A kit according to claim 9, wherein the T4EndoVII endonuclease and mismatch endonuclease are comprised in an enzyme composition, wherein
(a) the enzyme composition is cell-free,
(b) the enzyme composition has a temperature of 30° C. to 45° C.,
(c) the enzyme composition comprises ≥1 unit of T4EndoVII per μL and ≥1 unit of μL of the mismatch endonuclease,
(d) the enzyme composition further comprises a buffering agent, or
(e) the mismatch endonuclease is EndoMS.

11. A kit according to claim 9, wherein the mismatch endonuclease is EndoMS.

12. A method of DNA authentication comprising:
contacting
(a) dsDNA molecules comprising (i) heteroduplex dsDNA comprising at least one error and (ii) homoduplex dsDNA,
(b) a T4EndoVII endonuclease, and
(c) a mismatch endonuclease
to form authentication products corresponding to cleavage of the heteroduplex dsDNA at the error(s).

13. A method according to claim 12, further comprising forming the dsDNA molecules by denaturing and reannealing a source dsDNA to produce the dsDNA molecules.

14. A method according to claim 12, wherein the mismatch endonuclease is EndoMS.

15. A method according to claim 12, wherein the T4EndoVII and mismatch endonuclease are comprised in an enzyme composition, wherein
(a) the enzyme composition is cell-free,
(b) the enzyme composition has a temperature of 30° C. to 45° C.,
(c) the enzyme composition comprises ≥1 unit of T4EndoVII per μL and ≥1 unit of μL of the mismatch endonuclease,
(d) the enzyme composition further comprises a buffering agent, or
(e) the mismatch endonuclease is EndoMS.

16. A method according to claim 12, wherein the dsDNA fragments further comprise at least one homoduplex error-free dsDNA fragment, and wherein the T4Endo VII and mismatch endonuclease cleave the homoduplex error-free dsDNA fragment at a rate ≤80 mol. %.

17. A method according to claim 12, wherein the cleaved dsDNA substrates correspond in length (±5 nucleotides or less) to the number of nucleotides from (a) a 5′ end of the dsDNA substrate to the error, (b) from one error to an adjacent error, or (c) from the error to a 3′ end of the dsDNA substrate.

18. A method according to claim 13, wherein the source dsDNA comprises PCR amplicons from a base-edited genome or a chemically synthesized oligonucleotide, or DNA purified from a phage or cell.

19. A method according to claim 12, wherein the authentication products comprise ≤90 mol. %, ≤80 mol. %, ≤70 mol. %, ≤60 mol. %, ≤50 mol. %, ≤40 mol. %, ≤30 mol. %, ≤20 mol. %, ≤10 mol. %, ≤5 mol. %, ≤3 mol. %, or ≤1 mol. % of the heteroduplex dsDNA in the dsDNA molecules prior to contact with the endonucleases.

20. A method of forming screening products comprising:
performing DNA authentication according to claim 12 to form authentication products comprising dsDNA fragments;
amplifying the authentication products to form amplification products;
transforming bacteria with the amplification products to form transformation products; and
screening the transformation products to form screening products.

21. A method according to claim 20, wherein amplifying the authentication products comprising PCR amplification in a mixture comprising a polymerase, nucleotide triphosphates (NTPs), and primers.

22. A method according to claim 21, wherein forming the amplification products and amplifying the amplification products occurs in the same mixture, the mixture comprising the T4Endo VII, the mismatch endonuclease, the polymerase, the NTPs, and the primers.

23. A method according to claim 22, wherein the amplification products have ≤90%, ≤80%, ≤70%, ≤60%, ≤50%, ≤40%, ≤30%, ≤20%, ≤10%, ≤5%, ≤3%, or ≤1% as many errors as a corresponding quantity of amplification products arising from the same starting dsDNA molecules not contacted with the T4EndoVII or the mismatch endonuclease.

24. A method according to claim 20, wherein transforming comprises assembling the dsDNA fragments into a vector and transforming a competent bacteria with the vector.

25. A method according to claim 20, wherein screening the transformation products comprises plating the bacteria on agar and selecting individual colonies, which form the screening products.

26. A method according to claim 25, wherein the screening products comprise a fraction of error-free clones to total clones that is 1.1×, ≥1.2×, ≥1.4×, ≥1.6×, ≥1.8×, ≥2.0×, or ≥2.5× higher than the fraction error-free clones of a corresponding screen of non-authenticated dsDNA molecules.

27. A method of DNA fragment analysis comprising:
performing DNA authentication according to any one of claim 11 to form authentication products comprising dsDNA fragments;
analyzing the authentication products to determine the amount of cleaved dsDNA substrates in the authentication products and the amount of uncleaved dsDNA substrates in the authentication products; and
determining the proportion of heteroduplex dsDNA in the authentication products, wherein the proportion of heteroduplex dsDNA equals the amount cleaved dsDNA substrates divided by the amount of cleaved dsDNA substrates plus the amount of uncleaved dsDNA substrates.

28. A method according to claim 27, wherein analyzing comprises gel electrophoresis or microfluidics electrophoresis.

29. A method according to claim 27, wherein analyzing comprises determining the moles of uncleaved dsDNA and the moles of cleaved dsDNA.

30. A method according to claim 27, wherein the proportion of heteroduplex dsDNA equals the proportion of errors in the source dsDNA.
